(19) United States
(12) Patent Application Publication
(10) Pub. No.: US 2021/0212929 A1
(43) Pub. Date: Jul. 15, 2021

(54) ORAL FORMULATIONS FOR MITIGATING ACUTE CANNABIS INTOXICATION

(71) Applicant: Resurgent Pharmaceuticals, Inc., Minneapolis, MN (US)

(72) Inventor: Eric Greenbaum, Minneapolis, MN (US)

(21) Appl. No.: 17/150,385

(22) Filed: Jan. 15, 2021

Related U.S. Application Data
(60) Provisional application No. 62/961,503, filed on Jan. 15, 2020.

(51) Int. Cl. A61K 9/00; A61K 36/67; A61K 31/05; A61K 47/36; A61K 47/26; A61K 9/20

(52) U.S. Cl. CPC A61K 9/006; A61K 36/67; A61K 31/05; A61K 9/205; A61K 47/36; A61K 47/26; A61K 9/2018; A61K 9/0056

(57) ABSTRACT

Materials and method for mitigating the effects of acute cannabis intoxication are provided. For example, the present disclosure describes oral formulations comprising a peppercorn, a peppercorn substitute, or an extract thereof, a pharmaceutically acceptable excipient; and optionally CBD for use alleviating one or more unpleasant side effects of acute cannabis intoxication.

### BACKGROUND OF THE INVENTION

As more and more states legalize cannabis for medical and recreational purposes, its use continues to proliferate. Cannabinoids used alone or in combination have shown a variety of significant biological effects including but not limited to pain relief, anti-cancer, anti-inflammatory, anti-emetic, anti-convulsant, and many other therapeutic and recreational effects. While cannabis and the cannabinoids have remarkable safety profiles, acute cannabis intoxication is associated with a variety of unpleasant side effects such as anxiety or paranoia. For example, delta-9-tetrahydrocannabinol (THC), the primary psychoactive chemical of cannabis, is a promising therapeutic candidate for the treatment of chronic pain, but analgesia is accompanied by intoxication and abuse liability. Relieving intoxicating effects of cannabis could increase its therapeutic index. Thus, there is a need for new products to alleviate unpleasant side effects of acute cannabis intoxication.

### SUMMARY

The present disclosure describes formulations for use preventing, treating or minimizing an unpleasant effect of acute cannabis intoxication.

In a first aspect, the present disclosure provides an oral formulation for mitigating the effects of acute cannabis intoxication, the formulation including a peppercorn, a peppercorn substitute, or an extract thereof; a pharmaceutically acceptable excipient; and optionally cannabidiol (CBD). The peppercorn can be a black peppercorn. The oral formulation can include about 0.01 mg to 500 mg CBD. The oral formulation can further include a non-intoxicating cannabinoid. The quantity of peppercorn, peppercorn substitute or extract thereof in the oral formulation can be selected to achieve a predetermined amount of terpenoid, optionally a predetermined amount of β-caryophyllene. The oral formulation can be an oral spray. The oral formulation can be a compressed tablet. The oral formulation can be a candy-style lozenge. The oral formulation can further include at least one sugar alcohol. The oral formulation can further include a hydrocolloid selected from the group consisting of gelatin, gum acacia, carob gum, carrageenan, ghatti gum, guar gum, karaya gum, pectin, tragacanth gum, locust bean gum, xanthan gum and combinations thereof. The pharmaceutically acceptable excipient can be selected from the group consisting of emulsifiers, surfactants, colorants, flavorants, taste modifiers, buffering agents, pH adjusting agents, stabilizers, sweeteners and combinations thereof.

In a second aspect, the present disclosure provides a method of mitigating the effects of acute cannabis intoxication in a subject in need thereof, the method comprising administering to the subject an oral formulation including a peppercorn, a peppercorn substitute, or an extract thereof; a pharmaceutically acceptable excipient; and optionally CBD. The peppercorn can be a black peppercorn. The oral formulation can include about 0.01 mg to 500 mg CBD. The oral formulation can include a quantity of peppercorn, peppercorn substitute or extract thereof to achieve a predetermined amount of terpenoid, optionally a predetermined amount of pinene, piperine, or β-caryophyllene. The oral formulation can be administered as an oral spray, a compressed tablet, or a candy-style lozenge. The oral formulation can further include at least one sugar alcohol. The oral formulation can further include a hydrocolloid selected from the group consisting of gelatin, gum acacia, carob gum, carrageenan, ghatti gum, guar gum, karaya gum, pectin, tragacanth gum, locust bean gum, xanthan gum and combinations thereof. The pharmaceutically acceptable excipient can be selected from the group consisting of emulsifiers, surfactants, colorants, flavorants, taste modifiers, buffering agents, pH adjusting agents, stabilizers, sweeteners and combinations thereof. The oral formulation can be administered after the subject has used cannabis or administered concurrently with cannabis use.

### DETAILED DESCRIPTION

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from context.

### A. Cannabinoids

The cannabinoids are a class of molecules primarily obtained through the extraction of cannabis plant material, although synthetic and/or bioreactor production, and or other methods may also be used to obtain or modify them. The cannabinoids include, but are not limited to, THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), and CBT (cannabicitran). Naturally occurring cannabinoids may also be characterized by their type, such as Cannabigerol-type (CBG), Cannabichromene-type (CBC), Cannabidiol-type (CBD), Cannabinodiol-type (CBND), Tetrahydrocannabinol-type (THC), Cannabinol-type (CBN), Cannabitriol-type (CBT), Cannabielsoin-type (CBE), the Isocannabinoids, Cannabicyclol-type (CBL), Cannabicitran-type (CBT), and Cannabichromanone-type (CBCN). The chemical structures of exemplary cannabinoids organized by type are provided in TABLE 1.

TABLE 1
Chemical Structures of Exemplary Cannabinoids
Cannabigerol-type (CBG)

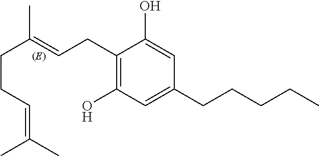 [SMILES: CCCCCc1cc(O)c(C/C=C(\C)CCC=C(C)C)c(O)c1]

Cannabigerol
(E)-CBG-C₅

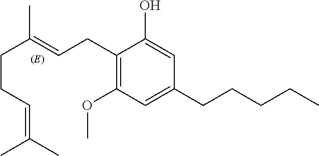 [SMILES: CCCCCc1cc(O)c(C/C=C(\C)CCC=C(C)C)c(OC)c1]

Cannabigerol
monomethyl ether
(E)-CBGM-C₅

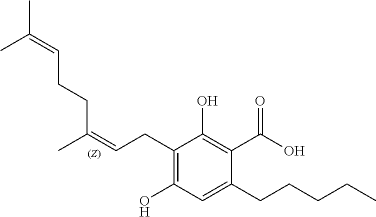 [SMILES: CCCCCc1cc(O)c(C/C=C(/C)CCC=C(C)C)c(O)c1C(=O)O]

Cannabinerolic acid A
(Z)-CBGA-C₅ A

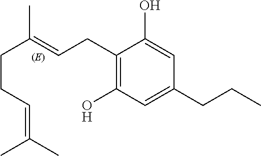 [SMILES: CCCc1cc(O)c(C/C=C(\C)CCC=C(C)C)c(O)c1]

Cannabigerovarin
(E)-CBGV-C₃

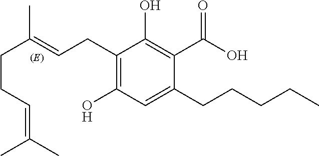 [SMILES: CCCCCc1cc(O)c(C/C=C(\C)CCC=C(C)C)c(O)c1C(=O)O]

Cannabigerolic acid A
(E)-CBGA-C₅ A

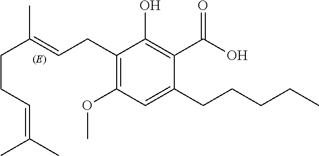 [SMILES: CCCCCc1cc(OC)c(C/C=C(\C)CCC=C(C)C)c(O)c1C(=O)O]

Cannabigerolic acid A
monotnethyl ether
(E)-CBGAM-C₅ A

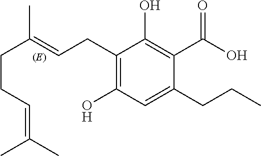 [SMILES: CCCc1cc(O)c(C/C=C(\C)CCC=C(C)C)c(O)c1C(=O)O]

Cannabigerovarinic acid A
(E)-CBGVA-C₃ A
Cannabichromene-type (CBC)

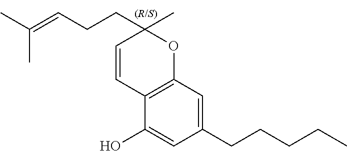 [SMILES: CCCCCc1cc(O)c2c(c1)OC(C)(CCC=C(C)C)C=C2]

(±)-Cannabichromene
CBC-C₅

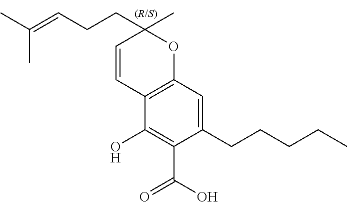 [SMILES: CCCCCc1cc2c(c(O)c1C(=O)O)C=CC(C)(CCC=C(C)C)O2]

(±)-Cannabichromenic acid A
CBCA-C₅ A

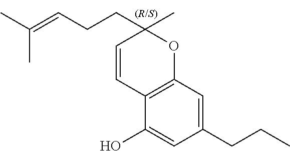 [SMILES: CCCc1cc(O)c2c(c1)OC(C)(CCC=C(C)C)C=C2]

(±)-Cannabivarichromene,
(±)-Cannabichromevarin
CBCV-C₃

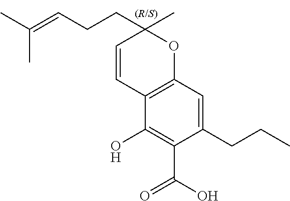 [SMILES: CCCc1cc2c(c(O)c1C(=O)O)C=CC(C)(CCC=C(C)C)O2]

(±)-Cannabichromevarinic acid A
CBCVA-C₃ A
Cannabidiol-type (CBD)

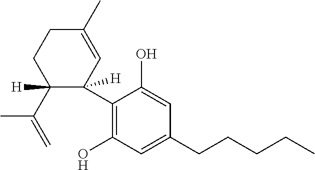 [SMILES: C=C(C)[C@@H]1CCC(C)=C[C@H]1c1c(O)cc(CCCCC)cc1O]

(−)-Cannabidiol
CBD-C₅

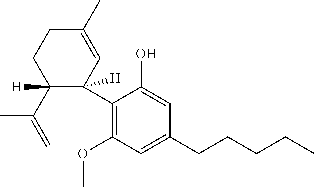 [SMILES: C=C(C)[C@@H]1CCC(C)=C[C@H]1c1c(O)cc(CCCCC)cc1OC]

Cannabidiol
momomethyl ether
CBDM-C₅

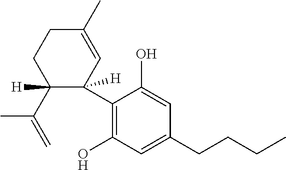 [SMILES: C=C(C)[C@@H]1CCC(C)=C[C@H]1c1c(O)cc(CCCC)cc1O]

Cannabidiol-C₄
CBD-C₄

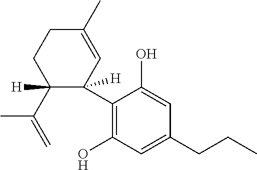 [SMILES: C=C(C)[C@@H]1CCC(C)=C[C@H]1c1c(O)cc(CCC)cc1O]

(−)-Cannabidivarin
CBDV-C₃

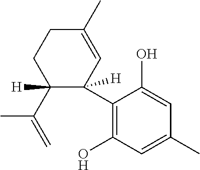 [SMILES: C=C(C)[C@@H]1CCC(C)=C[C@H]1c1c(O)cc(C)cc1O]

Cannabidiorcol
CBD-C₁

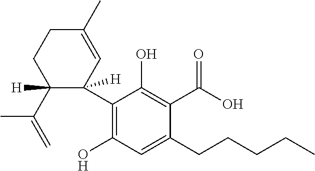 [SMILES: C=C(C)[C@@H]1CCC(C)=C[C@H]1c1c(O)cc(CCCCC)c(C(=O)O)c1O]

Cannabidiolic acid
CBDA-C₅

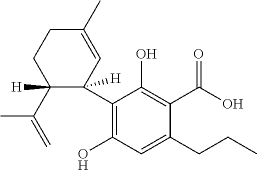 [SMILES: C=C(C)[C@@H]1CCC(C)=C[C@H]1c1c(O)cc(CCC)c(C(=O)O)c1O]

Cannabidivarinic acid
CBDVA-C₃
Cannabinodiol-type (CBND)

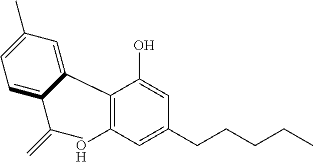

Cannabinodiol
CBND-C₅

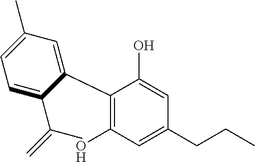

Cannabinodivarin
CBND-C₃
Tetrahydrocannabinol-type (THC)

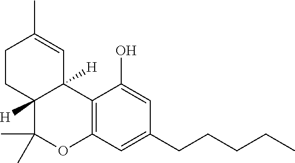 [SMILES: CCCCCc1cc(O)c2c(c1)OC(C)(C)[C@@H]1CCC(C)=C[C@@H]21]

Δ⁹-Tetrahydrocannabinol
Δ⁹-THC-C₅

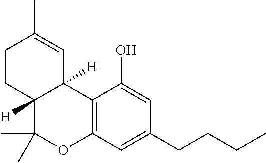 [SMILES: CCCCc1cc(O)c2c(c1)OC(C)(C)[C@@H]1CCC(C)=C[C@@H]21]

Δ⁹-Tetrahydrocannabinol-C₄
Δ⁹-THC-C₄

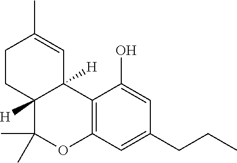 [SMILES: CCCc1cc(O)c2c(c1)OC(C)(C)[C@@H]1CCC(C)=C[C@@H]21]

Δ⁹-Tetrahydrocannabivarin
Δ⁹-THCV-C₃

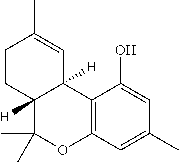

Δ⁹-Tetrahydrocannabiorcol
Δ⁹-THCO-C₁

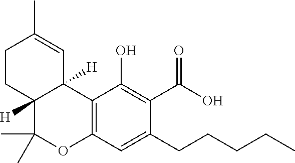 [SMILES: CCCCCc1cc2c(c(O)c1C(=O)O)[C@@H]1C=C(C)CC[C@H]1C(C)(C)O2]

Δ⁹-Tetrahydrocannabinolic acid A
Δ⁹-THCA-C₅ A

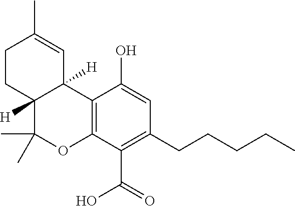 [SMILES: CCCCCc1cc(O)c2c(c1C(=O)O)OC(C)(C)[C@@H]1CCC(C)=C[C@@H]21]

Δ⁹-Tetrahydrocannabinolic acid B
Δ⁹-THCA-C₅ B

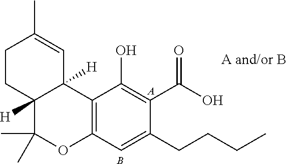

Δ⁹-Tetrahydrocannabinolic acid-C₄
A and/or B
Δ⁹-THCA-C₄ A and/or B

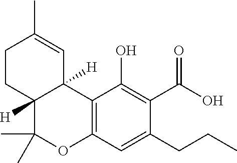 [SMILES: CCCc1cc2c(c(O)c1C(=O)O)[C@@H]1C=C(C)CC[C@H]1C(C)(C)O2]

Δ9-Tetrahydrocannabivarinic acid A
Δ⁹-THCVA-C₃ A

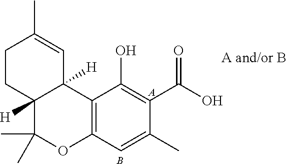

Δ⁹-Tetrahydrocannabiorcolic acid
A and/or B
Δ⁹-THCOA-C₁ A and/or B

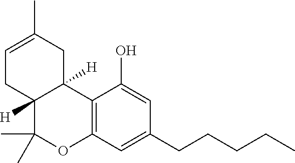 [SMILES: CCCCCc1cc(O)c2c(c1)OC(C)(C)[C@@H]1CC=C(C)C[C@@H]21]

(−)-Δ⁸-trans-(6aR,10aR)-
Δ⁸-Tetrahydrocannabinol
Δ⁸-THC-C₅

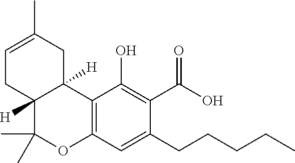 [SMILES: CCCCCc1cc2c(c(O)c1C(=O)O)[C@@H]1CC(C)=CC[C@H]1C(C)(C)O2]

(−)-Δ⁸-trans-(6aR,10aR)-
Tetrahydrocannabinolic acid A
Δ⁸-THCA-C₅ A

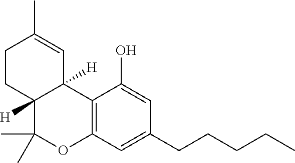 [SMILES: CCCCCc1cc(O)c2c(c1)OC(C)(C)[C@@H]1CCC(C)=C[C@@H]21]

(−)-(6as,10aR)-Δ⁹-
Tetrahydrocannabinol
(−)-cis-Δ⁹-THC-C₅
Cannabinol-type (CBN)

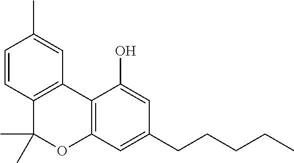 [SMILES: CCCCCc1cc(O)c2c(c1)OC(C)(C)c1ccc(C)cc1-2]

Cannabinol
CBN-C₅

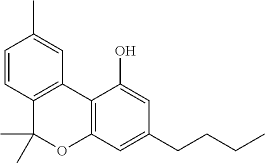 [SMILES: CCCCc1cc(O)c2c(c1)OC(C)(C)c1ccc(C)cc1-2]

Cannabinol-C₄
CBN-C₄

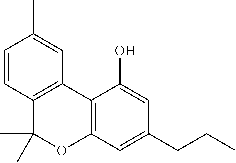 [SMILES: CCCc1cc(O)c2c(c1)OC(C)(C)c1ccc(C)cc1-2]

Cannabivarin
CBN-C₃

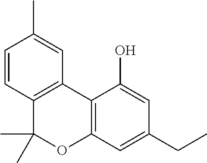 [SMILES: CCc1cc(O)c2c(c1)OC(C)(C)c1ccc(C)cc1-2]

Cannabinol-C₂
CBN-C₂

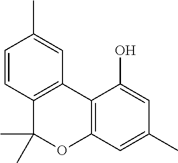

Cannabiorcol
CBN-C₁

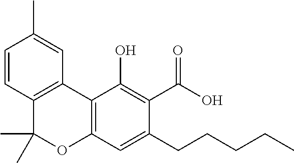 [SMILES: CCCCCc1cc2c(c(O)c1C(=O)O)-c1cc(C)ccc1C(C)(C)O2]

Cannabinolic acid A
CBNA-C₅ A

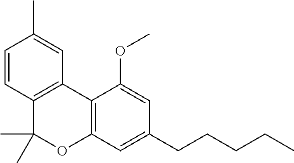 [SMILES: CCCCCc1cc(OC)c2c(c1)OC(C)(C)c1ccc(C)cc1-2]

Cannabinol methyl ether
CBNM-C₅
Cannabitriol-type (CBT)

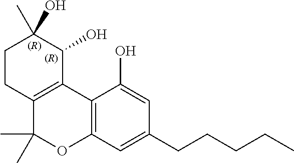

(−)-(9R,10R)-trans-Cannabitriol
(−)-trans-CBT-C₅

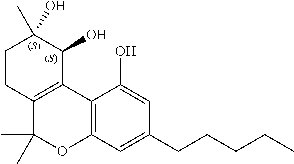

(+)-(9S,10S)-Cannabitriol
(+)-trans-CBT-C₅

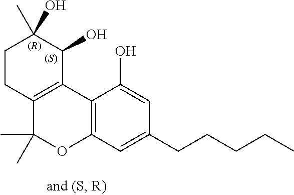

(±)-(9R,10S/9S,10R)-Cannabitriol
(±)-cis-CBT-C₅

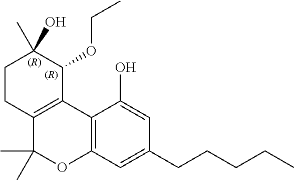

(−)-(9R,10R)-trans-
10-O-Ethyl-cannabitriol
(−)-trans-CBT-OEt-C₅

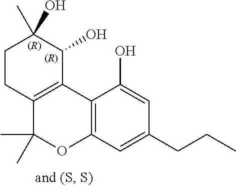

(±)-(9R,10R/9S,10S)-
Cannabitriol-C₃
(±)-trans-CBT-C₃

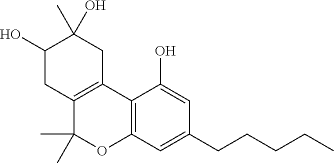 [SMILES: CCCCCc1cc(O)c2c(c1)OC(C)(C)C1=C2CC(C)(O)C(O)C1]

8,9-Dihydroxy-
Δ^{6a(10a)}-tetrahydrocannabinol
8,9-Di-OH-CBT-C₅

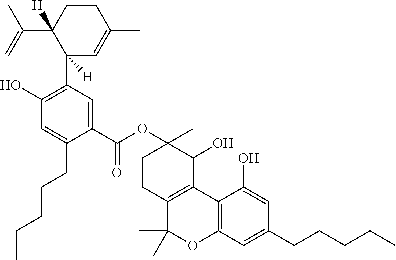 [SMILES: C=C(C)[C@@H]1CCC(C)=C[C@H]1c1cc(C(=O)OC2(C)CCC3=C(c4c(O)cc(CCCCC)cc4OC3(C)C)C2O)c(CCCCC)cc1O]

Cannabidolic acid A
cannabitriol ester
CBDA-C₅ 9-OH-CBT-C₅ ester

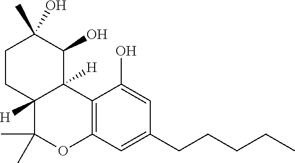 [SMILES: CCCCCc1cc(O)c2c(c1)OC(C)(C)[C@@H]1CC[C@](C)(O)[C@@H](O)[C@@H]21]

(−)-(6aR,9S,10S,10aR)-
9,10-Dihydroxy-hexahydro-
cannabinol, Cannabiripsol
Cannabiripsol-C₅

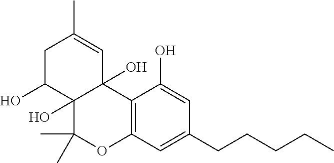 [SMILES: CCCCCc1cc(O)c2c(c1)OC(C)(C)C1(O)C(O)CC(C)=CC21O]

(−)-6a,7,10a-Trihydroxy-Δ⁹-
tetrahydrocannabinol
(−)-Cannabitetrol

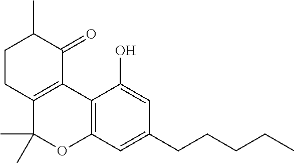 [SMILES: CCCCCc1cc(O)c2c(c1)OC(C)(C)C1=C2C(=O)C(C)CC1]

10-Oxo-Δ^{6a(10a)}-tetrahydrocannabinol
OTHC
Cannabielsoin-type (CBE)

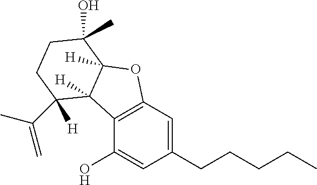

(5aS,6S,9R,9aR)-Cannabielsoin
CBE-C₅

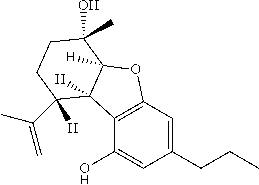

(5aS,6S,9R,9aR)-C₃-Cannabielsoin
CBE-C₃

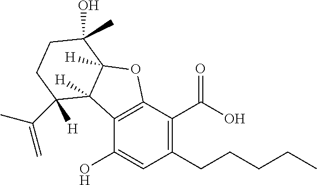

(5aS,6S,9R,9aR)-Cannabielsoic acid A
CBEA-C₅ A

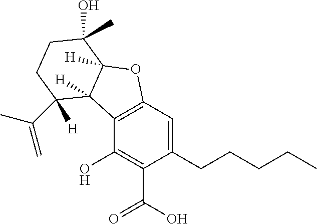

(5aS,6S,9R,9aR)-Cannabielsoic acid B
CBEA-C₅ B

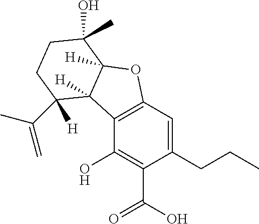

(5aS,6S,9R,9aR)-C₃-Cannabielsoic acid B
CBEA-C₃ B

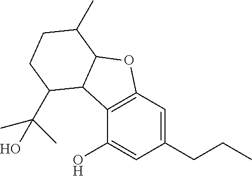 [SMILES: CCCc1cc(O)c2c(c1)OC1C(C)CCC(C(C)(C)O)C21]

Cannabiglendol-C₃
OH-iso-HHCV-C₃

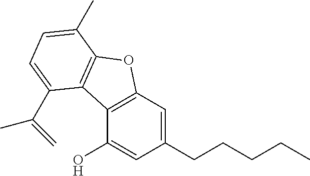 [SMILES: C=C(C)c1ccc(C)c2oc3cc(CCCCC)cc(O)c3c12]

Dehydro-
cannabifuran
DCBF-C₅

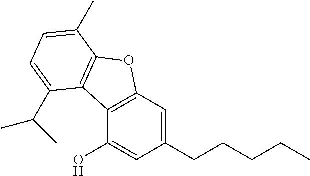 [SMILES: CCCCCc1cc(O)c2c(c1)oc1c(C)ccc(C(C)C)c12]

Cannabifuran
CBF-C₅
Isocannabinoids

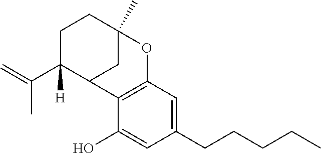 [SMILES: C=C(C)[C@@H]1CC[C@]2(C)CC1c1c(O)cc(CCCCC)cc1O2]

(−)-Δ⁷-trans-(1R,3R,6R)-
Isotetrahydrocannabinol

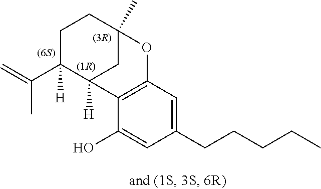 [SMILES: C=C(C)[C@H]1CC[C@]2(C)C[C@H]1c1c(O)cc(CCCCC)cc1O2]

(±)-Δ⁷-1,2-cis-(1R,3R,6S/1S,3S,6R)-
Isotetrahydrocannabivarin

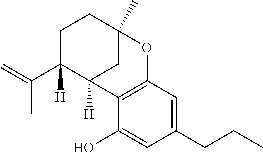 [SMILES: C=C(C)[C@@H]1CC[C@]2(C)C[C@H]1c1c(O)cc(CCC)cc1O2]

(−)-Δ⁷-trans-(1R,3R,6R)-
Isotetrahydrocannabivarin
Cannabicyclol-type (CBL)

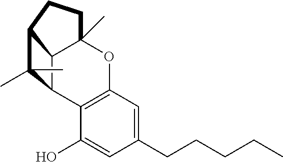

(±)-(1aS,3aR,8bR,8cR)
Cannabicyclol
CBL-C₅

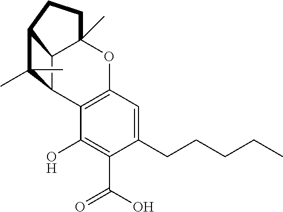

(±)-(1aS,3aR,8bR,8cR)
Cannabicyclolic acid A
CBLA-C₅ A

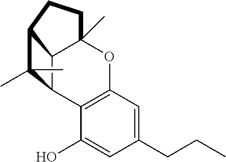

(±)-(1aS,3aR,8bR,8cR)-
Cannabicyclovarin
CBLV-C₃
Cannabicitran-type (CBT)

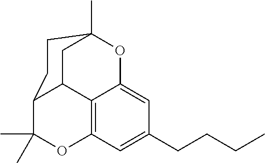 [SMILES: CCCCc1cc2c3c(c1)OC(C)(C)C1CCC(C)(CC31)O2]

Cannabicitran
CBT-C₅
Cannabichromanone-type (CBCN)

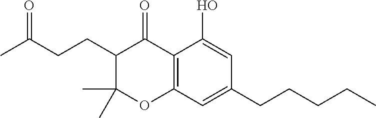 [SMILES: CCCCCc1cc(O)c2c(c1)OC(C)(C)C(CCC(C)=O)C2=O]

Cannabichromanone
CBCN-C₅

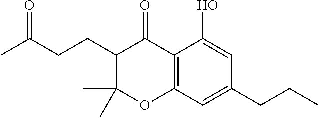 [SMILES: CCCc1cc(O)c2c(c1)OC(C)(C)C(CCC(C)=O)C2=O]

Cannabichromanone-C₃
CBCN-C₃

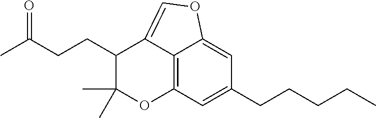 [SMILES: CCCCCc1cc2c3c(coc3c1)C(CCC(C)=O)C(C)(C)O2]

Cannabicoumaronone
CBCON-C₅

In addition to the naturally occurring cannabinoids, synthetic cannabinoids and derivatives thereof with cannabinomimetic properties, are available. Exemplary synthetic cannabinoids include, but are not limited to rimonabant, JWH-018, JWH-073, CP-55940; dimethylheptyran; HU-210; HU-331, SR144528, WIN 55,212-2, JWH-133, Levonantradol (Nantrodolum), AM-2201, etc.

Cannabinoids may be processed to improve hydrophilicity using any methods known to those having skill in the art, including but not limited to the creation of emulsions, microemulsions, nanoemulsions, encapsulation, complexation with solubility enhancers such as, for example, with a cyclodextrin, including but not limited to beta cyclodextrins, sulfobutylether beta cyclodextrins and the like; and complexation with sugar alcohols such as, for example erythritol, isomalt and the like.

### B. Active Agents for Mediating Effects of Cannabis Intoxication

CBD may ameliorate adverse effects of THC. In particular, a high dose of CBD (e.g., about 400 mg) can reduce the intoxicating effects of THC. In some cases, prolonged administration of high doses of CBD (e.g., 200 mg/day for 10 weeks) can reduce intoxication experienced from cannabis use. Compositions of the present disclosure can include 0.1-500 mg CBD.

Peppercorns (i.e., drupes of Piper nigrum), and black pepper substitutes contain various terpenoids that may alleviate one or more symptoms associated with cannabinoid intoxication. For example, β-caryophyllene (BCP), a sesquiterpene hydrocarbon that selectively binds to the cannabinoid type 2 receptor (CB2). BCP has anxiolytic-like and anti-depressant effects. Other terpenoids present in these plants include piperine, pinene, sabinene, limonene, pinene, myrcene, caryophyllene, phellandrene, and piperlonguminine. Fresh or dried, whole or ground drupes of Piper nigrum or peppercorn substitutes, or an extract thereof, can be administered to a subject in need of alleviation of an undesirable symptom of cannabis intoxication. A suitable amount of peppercorn or an extract thereof can vary between e.g., about 0.1 mg to about 10 g per dose, such as about 750 mg to 5 g, 500 mg to 1 g, 1 mg-100 mg, about 5 mg-75 mg, or about 10 mg-50 mg per dose.

Black pepper: Black pepper is produced from the still-green, unripe drupe of the black pepper vine, Piper nigrum. The drupes are cooked briefly in hot water, both to clean them and to prepare them for drying. The heat ruptures cell walls in the pepper, speeding the work of browning enzymes during drying. The drupes dry in the sun or by machine for several days, during which the pepper skin around the seed shrinks and darkens into a thin, wrinkled black layer. Once dry, the spice is called black peppercorn. Once the peppercorns are dried, pepper spirit and oil can be extracted from the berries by crushing them. Pepper spirit is used in many medicinal and beauty products. Pepper oil is also used as an ayurvedic massage oil and in certain beauty and herbal treatments.

White pepper: White pepper consists solely of the seed of the ripe fruit of the black pepper vine, with the thin darker-colored skin (flesh) of the fruit removed. This is usually accomplished by a process known as retting, where fully ripe red pepper berries are soaked in water for about a week so the flesh of the peppercorn softens and decomposes; rubbing then removes what remains of the fruit, and the naked seed is dried. Sometimes alternative processes are used for removing the outer pepper from the seed, including removing the outer layer through mechanical, chemical, or biological methods. Ground white pepper is used commonly in Chinese, Thai and Portuguese cuisine, but also in salads, light-colored sauces, and mashed potatoes as a substitute, because black pepper would visibly stand out. However, white pepper lacks certain compounds present in the outer layer of the drupe, resulting in a different overall flavor.

Green pepper: green pepper, like black pepper, is made from unripe drupes. Dried green peppercorns are treated to retain the green color, such as with sulfur dioxide, canning, or freeze-drying. Pickled peppercorns, also green, are unripe drupes preserved in brine or vinegar. Fresh, unpreserved green pepper drupes are used in some Asian cuisines, particularly Thai cuisine. Their flavor has been described as “spicy and fresh”, with a “bright aroma.”

Red peppercorns: Red peppercorns are usually obtained by harvesting ripe drupes of the black pepper vine, and preserving these in brine and vinegar. Ripe red drupes can also be dried using the same color-preserving techniques used to produce green peppercorns.

Pink pepper: Pink peppercorns are the fruits of the Peruvian pepper tree, Schinus molle, or its relative, the Brazilian pepper tree, Schinus terebinthifolius, plants from a different family (Anacardiaceae).

Peppercorn substitutes: The bark of Drimys winteri (“canelo” or “winter's bark”) is used as a substitute for pepper in cold and temperate regions of Chile and Argentina, where it is easily found and readily available. In New Zealand, the seeds of kawakawa (Macropiper excelsum), a relative of black pepper, are sometimes used as pepper; the leaves of Pseudowintera colorata (“mountain horopito”) are another replacement for pepper. Several plants in the United States are also used as pepper substitutes, such as field pepperwort, least pepperwort, shepherd's purse, horseradish, and field pennycress. A suitable amount of peppercorn substitute or an extract thereof can vary between e.g., about 10 mg to about 1 g per dose.

Specific terpenes can also be used in the methods of the present disclosure. For example, terpenoids such as piperine, pinene, and caryophyllene can be isolated from a suitable botanical source, or synthesized, and added to the CBD- or peppercorn-containing formulation.

### C. Other Active Ingredients

Formulations of the present disclosure can include an essential oil as an additional active ingredient. Suitable essential oils can be selected from Sweet Orange (Citrus sinensis spp), Peppermint (Mentha piperita spp), Lemon (Citrus limon spp), Lavender (Lavendula angustifolia spp), Vanilla (Vanilla planifolia spp), and combinations thereof. In some embodiments, the formulation includes a cannabis oil extract including Vitamin E and one or more essential oils selected from Sweet Orange (Citrus sinensis spp), Peppermint (Mentha piperita spp), Lemon (Citrus limon spp), Lavender (Lavendula angustifolia spp) and Vanilla (Vanilla planifolia spp).

Other essential oils that can be used in the compositions of the invention include, but are not limited to: Agarwood; Agarwood Attar; Ahibero; Allspice; Almond, bitter; Amber Oil; Ambrette Seed; Amyris; Angelica Root; Angelica Seed; Aniseed; Anise; Anise (star); Armoise (Mugwort); Artemisia vestita; Asafoetida; Bakul; Balsam of Peru Oil; Balsam of Peru Resin; Balsamite; Baobab Oil; Basil, Sweet ct Linalool; Basil, Sweet ct Linalool-Organic; Basil, Sweet ct Methyl Chavicol-Organic; Bay; Beeswax; Bergamot; Birch; Boldo; Boronia; Black Cumin; Black Currant Bud; Blue Lotus Attar; Broom; Buchu; Bupleurum (Bupleurum fruticosum); Buddha wood; Butter; Cabreuva; Cade; Cajuput; Calamus; Calendula; Camomile (or Chamomile); Camphor; Cananga; Cangerana; Cape Chamomile (Ericephalus punctulatus) S. Africa, Wild Harvest; Cape May; Caraway; Caraway; Cardamom; Carnation; Carrot Seed; Cascarilla; Cassia; Cassie; Catnip; Cedar (Cedrus) India; Cedarwood; Cedarwood, Atlas-Organic; Cedarwood, Himalayan; Cedarwood, Texas; Cedarwood, Virginia; Celery leaf; Celery Seed; Chamomile, Blue; Chamomile; Chamomile, Roman (Anthemis nobilis); Champa Attar (Michelia champaca) India; Champaca; Chaste tree; Cilantro; Cinnamon; Cinnamon Bark; Cistus; Cistus (Cistus ladaniferus) Corsica; Citronella; Clary Sage Absolute; Clary Sage, Bulgaria; Clary Sage, Russia; Clary Sage, USA; Clementine; Clove; Clove Bud; Cacao; Coconut Pulp; Coffee Bean Oil; Cognac, Green; Coleus; Combava (fruit or leaf); Copaiba; Coriander; Coriander Seed; Cucumber Hydrosol; Cumin; Cumin Seed; Cypress Leaf; Cypress, Blue; Davana; Dill; Elemi; Eucalyptus, Blue Gum; Eucalyptus, Blue Mallee; Eucalyptus, Lemon; Fennel (Foeniculum vulgare) Bulgaria; Fennel, Sweet; Fenugreek; Fern (sweet); Fleabane; Fir Needle; Fir, Balsam; Fir, Douglas; Fir, Silver; Fragonia; Frankincense, India; Frankincense, Somalia; Frankincense Frereana; Frankincense, Oman; Frankincense, Oman; Frankincense, Somalia; Galangal; Galbanum; Geranium; Geranium, Egypt; Geranium, Rose; Geranium, South Africa; Ghandi root; Ginger; Ginger Lily; Ginger, Fresh; Gingergrass (Cymbopogon martinii); Goldenrod; Grapefruit, Pink; Grapefruit, Ruby Red; Grapefruit, White; Hay; Helichrysum, Albania; Helichrysum, Croatia; Hina Attar, India; Hop; Hyssop Decumbens; Hyssop; Immortelle; Jasmine Absolute, Egypt; Jasmine Absolute, India; Jasmine Concréte; Jasmine; Jasmine Sambac; Jatamansi, (Nardostachs jatamansi); Juniper; Juniper Berry (Juniperus communis); Juniper Leaf/Berry; Kaffir Lime; Kava; Labdanum; Larch needle; Laurel (Laurus nobilis) Corsica; Laurel Leaf; Lavandin, Grosso; Lavender—High Elevation; Lavender—Wild; Lavender Absolute; Lavender Hydrosol; Lavender, Bulgaria; Lavender, France; Lavender, Maillette; Leleshwa; Lemon; Lemon Tea Tree; Lemon verbena; Lemongrass; Lentisque (Pistacia lentiscus) Corsica; Lime; Lime Essence Oil; Lime, Distilled; Liquidambar (Styrax); Longoza; Lotus Absolute, Pink; Lotus Absolute, White; Lovage leaf, Lovage root; Magnolia flower; Mandarin; Mandarin, Green; Mandarin, Red; Mandarin, Yellow; Mango ginger; Marjoram; Manila oil; Melissa; Mint; Mint, Himalayan (Mentha arvensis); Mitti Attar; Motia Attar (Jasmine sambac) India; Mugwort; Mustard; Myrrh; Myrtle, Green; Myrtle (Myrtus Communis); Nagarmotha (Cypriol); Neem (Azadirachta indica) India; Neroli; Niaouli; Nutmeg; Nut grass; Oakmoss Absolute; Oakwood; Opopanax, Sweet Myrrh (Commiphora guidotti); Orange, Blood; Orange, Sweet; Orange, Wild; Orange Blossom; Orange Essence Oil; Orange, Bitter Green; Orange, Bitter Red; Oregano; Orris Butter; Osmanthus Absolute; Palmarosa; Palmarosa, Nepal; Palmarosa, Sri Lanka; Palo Santo (Bursera graveolens); Palo Santo; Patchouli; Absolute; Patchouli, Dark; Patchouli, Light; Patchouli, Sri Lanka; Pennyroyal; Pepper, Black; Peppercorn, Pink; Peppermint, Chocolate; Peppermint, France; Peppermint, India; Peppermint, USA; Petitgrain Absolute; Petitgrain Bigarade; Petitgrain sur Fleurs; Petitgrain, Mandarin; Pimento; Pine; Pinion Juniper Co-distillation, Colorado, Wild Harvest; Pinon Pine (Pinus edulis) Colorado, Wild Harvest; Pitta blend (Lavender, Rose Geranium, Ruh Khus); Plai; Pomegranate Seed; Rhododendron (Rhododendron anthopogon); Rhododendron Leaf; Rosalina; Rose; Rose Attar; Rose de Mai Absolute; Rose de Mai Concrete; Rose de Mai Organic Extract; Rose geranium; Rose Hip Seed; Rose Otto, Bulgaria; Rose Otto, Turkey; Rose Otto, White-Organic; Rose vetiver; Rosemary Antioxidant; Rosemary ct Cineole; Rosemary ct Verbenone; Rosewood; Rue; Ruh Khus (Vetiveria zizaniodes); Saffron Attar, India; Sage; Samphire (Cristhmum maritimum) Corsica; Sandalwood; Sandalwood, New Caledonia; Sandalwood, Australian-Premium; Sandalwood (Santalum spicatum), Australia; Sandalwood Oil, Royal Hawaiian (Santalum paniculatum); Sandalwood, Royal Hawaiian; Sassafras; Savitri Rose Perfume; Sea Buckthorn; Seaweed; Sierra Juniper (Juniperus occidentalis); Spearmint; Spearmint (Mentha Spicata) Israel; Spikenard; Spikenard, Green; Spruce, Black; Spruce (Picea mariana) Canada; St. John's Wort 2; St. John's Wort (Hypericum perforatum) Bulgaria; Tagetes; Tamanu (Foraha) Oil; Tangelo; Tangerine; Tangerine Murcott; Tansy; Tansy, Blue; Tarragon; Tea Tree; Tea Tree (Leptospermum citratum), Lemon Scented; Tea Tree (Melaleuca alternifolia) South Africa; Thuja; Thyme; Thyme ct Linalool; Tobacco; Tonka Bean; Tuberose; Tulsi, Holy Basic Oil (Ocimum sanctum); Turmeric; Vanilla; Vanilla Bourbon; Verbena; Vetiver—Double Distilled; Vetiver, El Salvador; Vetiver, Haiti; Vetiver, Sri Lanka; Violet Leaf; White Fir (Abies concolor); White Lotus Attar; White Sage (Salvia apiana); Wild Carrot, Corsica; Wintergreen; Wintergreen; Yarrow; Yarrow, Blue; Ylang; Yuzu; and combinations thereof.

The compositions of the invention can also include one or more herbal extracts of Abas, Abele, Abies balsamea, Absinthe, Absinthium, Acacia, Acacia spp., Acai Berries, Acerola, Achillea Millefolium, Achiote, Aconite, Aconitum Napellus, Acorns, Acorns calamus, Acorns gramineus, Adansonia digitata, Adder's Mouth, Adderwort, Adiantum capillus-veneris, Aesculus Hippocastanum, Aframomum melegueta, African Geranium, African Ginger, Agastache foeniculum, Agave, Agnus Castus, Agrimonia Eupatoria, Agrimony, Agropyron Repens, Ague Grass, Ague Root, Ague Tree, Agueweed, Ajamoda, Ajave Seeds, Ajenjo, Ajowan, Ajuga Reptans, Ajvain, Ajwan, Ajwain, Akebia, Akebia quinata, Alaskan Ginseng, Alchemilla Vulgaris, Alchornea Species, Alder, Alder Buckthorn, Alder Dogwood, Alecost, Alehoof, Aletris, Aletris Farinosa, Alexandrian Laurel, Alexandrian Senna, Alfalfa, Algarroba, Alkanet, Allheal, Alligator Pepper, Allium cepa, Allium porrum, Allium sativum, Allium schoenoprasum, Allium tuberosum, Allspice, Almond, Alnus glutinosa, Alnus rubra, Aloe ferox, Aloeroot, Aloes, Aloe Vera, Aloysia triphylla, Alpine Strawberry, Alpinia Officinarum, Althaea, Althaea Officinalis, Aluka, Alumroot, Amara Aromatica, Amaracus, Amaranth, Amaranthus Hypocondriacus, Amber Touch-and-heal, Ambroise, Ambrose, Amburana, America-Hodoimo, American Aloe, American Angelica, American Ash, American Aspen, American Basswood, American Bayberry, American Bee Balm, American Beech, American Bugleweed, American Carob, American Cranesbill, American Cress, American Dill, American Dogwood, American Ginseng, American Ground Lily, American Groundnut, American Linden, American Mandrake, American Melissa, American Saffron, American Sanicle, American Sarsaparilla, American Sloe, American Spikenard, American Upland Cotton, American Valerian, American Winter Cress, American Wormroot, American Wormseed, Amla, Ammi Visnaga, Anacardium Occidentale, Ananas Comosus, Anchusa Officinalis, Andiroba, Andrographis, Andrographis paniculata, Anemone, Anemone Pulsatilla, Anemopsis californica, Anethum Graveolens, Angelica, Angelica Archangelica, Angelica Sinensis, Angelica Tree, Angostura, Angostura trifoliata, Anise, Aniseed, Aniseed Stars, Anise Fern, Anise Hyssop, Anise Plant, Annatto, Annona muricata, Annona reticulata, Annual Marjoram, Anthemis Nobilis, Anthoxanthum nitens, Anthriscus cerefolium, Antilles Cherry, Apios americana, Apium Graveolens, Apple, Apple Mint, Apple-of-Peru, Apricot Vine, Apsidium, Aralia Racemosa, Arbe a suif, Arberry, Arboloco, Arbor Vitae, Arbutus, Arbutus menziesii, Arbutus Uva Ursi, Archangel, Archangelica, Archangelica officinalis, Arctium Lappa, Arctostaphylos Uva Ursi, Ardraka, Argan, Argania, Argania spinosa, Argemone Mexicana, Argentine, Aristolochia serpentaria, Aristotelia chilensis, Aritha, Arjaka, Arjuna, Armoracia Rusticana, Armstrong, Arnica, Arnica Flowers, Arnica Montana, Arnica Root, Aromatic Sumac, Aromatic Wintergreen, Arrowroot, Artemisia, Artemisia Abrotanum, Artemisia Absinthium, Artemisia capillaris, Artemisia Dracunculus, Artemisia Tridentata, Artemisia Vulgaris, Artocarpus altilis, Artocarpus heterophyllus, Arugula, Asafoetida, Asclepias Tuberosa, Ascophyllum nodosum, Ash, Ashwaganda, Asian Ginseng, Aspalathus Linearis, Asparagus Cochinchinensis, Asparagus racemosus, Asparagus Root, Asperula Odorata, Aspilia, Aspilia Mossambicensis, Ass-ear, Asthma Plant, Asthma Weed, Astragalus, Astragalus Membranaceus, Atropa belladonna, Auld Wife's Huid, Autumn Crocus, Avena Sativa, Avens, Averrhoa carambola, Avocado, Ayak Chichira, Ayuk Willku, Azadirachta Indica, Azafran, Babchi Seeds, Bacc, Bachelor's-button, Bacopa Monniera, Bahama Berry, Baical Skullcap, Bai Guo, Bai Mu Erh, Ba Ji Tian, Baldina, Balinghoy, Ballota nigra, Balm Mint, Balm of Gilead, Balmony, Balsam Copaiba, Balsam Fir, Balsam of Gilead, Balsam of Peru, Balsam Tree, Bank Cress, Banisteriopsis caapi vine, Baobab, Baptisia, Baptisia Tinctoria, Barbados Aloe, Barbados Cherry, Barbarea verna, Barberry, Barbary Fig, Bardana, Barley, Barosma Betulina, Barren Strawberry, Baran, Basil, Basil Thyme, Basin Sagebrush, Basketbush, Basswood, Bastard Cardamom, Bastard Saffron, Bast Tree, Bauple Nut, Bayawas, Bayberry, Bayberry Bush, Bayberry Wax Tree, Bay Laurel, Beaked Parsley, Bean of India, Bean Trefoil, Bearberry, Bearbind, Beard Lichen, Bear's Foot, Bear's-grape, Bear's-paw, Bear's Weed, Beaumont Root, Beauty Leaf, Bee Balm, Bee Bread, Beech, Beechdrops, Beech Wheat, Bee Plant, Bee Sage, Bee's Nest, Beggar's Buttons, Belladonna, Belle Isle Cress, Bellyache Root, Benjamin Bush, Benzoin Gum, Benzoin Tree, Berberidis, Berberis Aquifolium, Berberis Vulgaris, Berberry, Bergamot Mint, Bergamot Orange, Bertholletia Excelsa, Betel, Bethroot, Betony, Betula alba, Betula pendula, Bhang, Bian Xu, Bible Hyssop, Bible-leaf, Big Sagebrush, Bilberry, Billy-goat Clover, Biltmore Ash, Bindweed, Bird's-foot, Bird's Nest, Birthroot, Birthwort, Biscuits, Bishop\'s Weed, Bistort, Bitter Aloe, Bitter Ash, Bitter Bark, Bitter Dock, Bitter Leaf, Bitter Melon, Bitter Nightshade, Bitter Orange, Bitter Orange Peel, Bitter Quassia, Bittersweet, Bitter Trefoil, Bitter Wood, Bitterworm, Bixa orellana, Black Alder, Black Alder Tree, Blackberry, Black Cherry, Black Choke, Black Chokeberry, Black Cohosh, Black Cohush, Blackcurrant, Black Dogwood, Black Ginger, Black Haw, Black Henbane, Black Horehound, Black Locust, Black Mustard, Black Pepper, Black Root, Black Sampson, Black Sanicle, Black Snakeroot, Black Stinking Horehound, Black Tany, Blackthorn, Black Thyme, Black Walnut, Black Whortleberry, Blackwort, Bladder Cherry, Bladder Fucus, Bladderpod, Bladderwrack, Blazing Star, Blessed Herb, Blessed Thistle, Blind Nettle, Bloodroot, Blood Vine, Bloodwort, Blooming Sally, Blow Ball, Blue Balm, Blueberry, Bluebottle, Blue Cohosh, Blue Curls, Blue Dandelion, Blue Flag, Blue Giant Hyssop, Blue Ginseng, Blue Gum, Blue Gum Tree, Blue Iris, Blue Mountain Tea, Blue Pimpernel, Blue Rocket, Blue-Sailors, Blue Skullcap, Blue Violet, Blunt-leaved Dock, Bodhi Tree, Bofareira, Bogbean, Bo He, Bola, Boldina, Boldo, Boldoa, Boldu, Boldus, Boneset, Bookoo, Borage, Borago Officinalis, Boswellia carteri, Bo-Tree, Bottlebrush, Bouncing Bet, Bourtree, Bowman's Root, Boxberry, Boxwood, Brahmi, Bramble, Brandy Mint, Brassica alba, Brassica juncea, Brassica nigra, Brassica oleracea, Brassica rapa Pekinensis, Brazilian Ginseng, Brazil Nut, Breadfruit, Bread Wheat, Bride's Button, Bridewort, Brigham Tea, Brindall Berry, Brindle Berry, Brinton Root, British Myrrh, Broad-leaved Dock, Bromelain, Brook Bean, Brooklime, Broom, Broom Flowers, Broom Tops, Broom Tea-Tree, Brown Mustard, Brownwort, Bruisewort, Bryonia Alba, Bryony, Buchu, Buckbean, Buckeye, Buckler-leaved Sorrel, Buckthorn, Buckwheat, Bucku, Buddha Fruit, Buffalo Herb, Bugbane, Bugbane Squawroot, Bugle, Bugleweed, Bugloss, Bu Gu Zhi, Bugwort, Bull Flower, Bullock's Heart, Bull's Heart, Bunny's Ears, Bupleurum, Bupleurum Chinense, Bur, Burage, Burdock, Burdock Burrs, Burren Myrtle, Burr Marigoldt, Burrs, Burr Seed, Bush Nut, Butcher's Broom, Butterbur, Butterfly Weed, Butternut, Butter Winter, Butterwort, Butterweed, Buttons, Caban Cherry, Cabbage, Cabbage Palm, Cabbage Rose, Cacao, Cacari, Cajeput Tree, Cajueiro, Calabar Bean, Calamint, Calamintha Nepeta, Calamus, Calendula, Calendula Officinalis, California Poppy, Calluna Vulgaris, Calophyllum inophyllum, Caltrop, Calumba, Cambodian Mint, Camel Grass, Cammellia Sinensis, Camocamo, Camphor, Camphor Tree, Camptotheca Acuminata, Camu, Canabis, Canada Balsam, Canada Root, Canada Tea, Canadian Fleabane, Canaigre, Cananga odorata, Cancerosa, Caner Root, Cancer Tree, Candle Berry, Cane Ash, Canistel Fruit, Cankerwort, Cannabis Sativa, Cape Aloe, Cape Gooseberry, Caperberry, Caperbush, Capers, Capon's Tail, Capparis spinosa, Capsaicin, Capsella Bursa-Pastoris, Capsicum, Capsicum Annuum, Capsicum chinense, Carambola, Carapa guianensis, Caraway, Caraway Seed, Cardamom, Cardamom Seeds, Cardamon, Carduus Marianus, Carica Papaya, Carob, Carolina Jasmine, Carom, Carony Bark, Carpenter's-herb, Carpenter's-square, Carpenter's Weed, Carrageen, Carrot, Carthamus Tinctorius, Carum Carvi, Cascara, Cascara Buckthorn, Cascara Sagrada, Caseweed, Cashew Nut Shells, Cassava, Cassia Senna, Castanea Sativa, Castor Bean Plant, Castor Oil Plant, Catalonian Jasmine, Catchweed, Catha, Catha Edulis, Catmint, Catnep, Catnip, Catnip, Cat's Claw, Cat's-foot, Cat's-play, Catswort, Cat Thyme, Catuaba, Caulophyllum Thalictroides, Cayenne, Ceanothus Americanus, Cedar Nut, Celandine, Celery, Centaurea Cyanus, Centaurium Erythraea, Centaury, Centella Asiatica, Century Plant, Cephaelis Ipecacuanha, Cerasee, Ceratonia Siliqua, Cereso, Cetraria Islandica, Chaga Mushroom, Chai Hu, Chamaelirium Luteum, Chamomile, Chanca Piedra, Chandan, Chang Pu, Chaparral, Charapilla, Chaste Berry, Chaste Tree, Chat, Chaulmoogra, Checkerberry, Cheeseflower, Cheese Rennet, Cheiranthus Cheiri, Chelidonium Majus, Chelone Glabra, Chenopodium Ambrosioides, Chen-Pi, Cherry Birch, Chervil, Chia, Chian, Chien, Chiang, Chickweed, Chicory, Chih-ma, Chi-hsueh-ts'ao, Chilean Wineberry, Chilgoza, Chili Pepper, Chimaphila Umbellata, China Root, Chin-ch'iao-mai, Chinese Angelica, Chinese Cabbage, Chinese Chives, Chinese Foxglove, Chinese Ginseng, Chinese Gold Thread, Chinese Lantern, Chinese Licorice, Chinese Mustard, Chinese Nettle, Chinese Star Anise, Chinese Wolfberry, Chink, Chionanthus Virginicus, Chirayata, Chiretta, Chittembark, Chives, Chocolate, Chocolate Root, Chocolate Vine, Choke Cherry, Chondrus Crispus, Christmas Tree, Chrysanthemum, Chrysanthemum Balsamita, Chrysanthemum Cinerariifolium, Chrysanthemum Morifolium, Chuan Xin Liang, Chuchupate, Church Steeples, Cicely, Cichorium Intybus, Cilantro, Cimicifuga, Cimicifuga Racemosa, Cinchona, Cinchona Bark, Cinchona spp, Cingulum Sancti Johannis, Cinnabar Root, Cinnamomum Camphora, Cinnamomum Zeylanicum, Cinnamon, Cinnamonwood, Cinquefoil, Cirsium Vulgare, Citroengrass, Citrus aurantium, Citrus bergamia, Citrus ichangensis×Citrus reticulata var. austera, Citrus limon, Citrus reticulata, Citrus thyme, City Avens, Clary, Clary Sage, Clear Eye, Cleavers, Clematis Stem, Clove, Clove Garlic, Clover, Clover Broom, Clove Root, Clown's Woundwort, Clubfoot Moss, Club Moss, Cnicus Benedictus, Coca, Coca Shrub, Cocashweed, Cochlearia Officinalis, Cocklebur, Cockle Buttons, Cocoa, Codonopsis, Codonopsis pilosula, Coffea Arabica, Coffee, Coffeeweed, Coicis, Coix, Coix Lachryma-jobi, Cola nitida, Colchicum, Colchicum Autumnale, Coleus, Coleus Forkolil, Coleus Forskohlii, Colewort, Colicroot, Colla, Collinsonia Canadensis, Colorado Cough Root, Coltsfoot, Colt's-tail, Comfrey, Commiphora Molmol, Commiphora Mukul, Commiphora Opobalsamum, Common Alder, Common Alkanet, Common Anise, Common Arnica, Common Ash, Common Balm, Common Barberry, Common Basil, Common Blue Violet, Common Broom, Common Buckthorn, Common Buckwheat, Common Bugle, Common Burnet, Common Caraway, Common Centaury, Common Chamomile, Common Club Moss, Common Cotton, Common Dock, Common Dill, Common Fennel, Common Fenugrec, Common Flax, Common Foxglove, Common Hazel, Common Holly, Common Hop, Common Horehound, Common Hyssop, Common Jasmine, Common Juniper, Common Lavender, Common Lime, Common Madder, Common Mallow, Common Marjoram, Common Nettle, Common Oats, Common Onion, Common Parsley, Common Periwinkle, Common Privet, Common Rue, Common Sage, Common Sagebrush, Common Sea-Buckthorn, Common Stinging Nettle, Common Strawberry, Common Sundew, Common Thistle, Common Thyme, Common Wheat, Common White Jasmine, Common Willow, Common Wormwood, Compass Plant, Compass Weed, Compositae, Conium Maculatum, Consormol, Consumptive's Weed, Convallaria Majalis, Convolvulus Sepium, Cool Tankard, Copaiba, Copal, Copaifera Species, Coptidis, Coptis, Coptis chinensis, Coptis Rhizome, Cordyceps, Cordyceps sinensis, Coriander, Coriandrum sativum, Corn, Cornelian Tree, Cornflower, Cornish Lovage, Corn Mint, Corn Poppy, Corn Rose, Cornsilk, Cornus Florida, Corsican Mint, Corsican Pepper, Corydalis, Corydalis Rhizome, Corydalis Yanhusuo, Corylus avellana, Costmary, Cotton, Cotton Thistle, Couch Grass, Coughroot, Coughweed, Coughwort, Countryman's-treacle, Cowbloom, Cow Chervil, Cow Clover, Cow Cress, Cowgrass, Cowplant, Cowslip, Crampbark, Crampweed, Cranberry, Cranberry Bush, Cranberry Tree, Cranesbill, Crataegus Monogyna, Crataegus Oxyacantha, Crataeva, Crataeva nurvula, Cream Of Tartar Tree, Creathnach, Creeping Charlie, Creeping Thyme, Creosote Bush, Crocus sativus, Crosswort, Croton Lechleri, Crowberry, Crow Com, Cuban Oregano, Cubeb Pepper, Cuckoo's Cap, Cucurbita Pepo, Culantrillo, Culver's Physic, Culver's Root, Cumaru, Cumaruzeiro, Cumin, Cuminum cyminum, Curacao Aloe, Curare, Curcuma longa, Curcuma zedoaria, Cure-all, Curled Dock, Curled Mint, Curly Parsley, Curry-leaf tree, Curry Tree, Cuscus, Cuscuta Epithymum, Cusparia Bark, Custard Apple, Cutleaf Bugleweed, Cutweed, Cydonia Oblonga, Cymbopogon Citratus, Cypripedium Pubescens, Da Huang, Dalcini, Dalmatian Iris, Dalmation Insect Flower, Dalmation Pellitory, Dalmatian Sage, Damiana, Dandelion, Dang Gui, Danish Dill, Dan Shen, Daruharidra, Da suan, Datura Stramonium, Daucus carota, Deadly Nightshade, Deadmen's Bells, Dead Nettle, Dead-Rat Tree, Death-flower, Deerberry, Desert Cactus, Desert Oregano, Desert Tea, Devil's-apple, Devil's Bit, Devil's-bones, Devil's Cherries, Devil's Claw, Devil's Club, Devil's Dung, Devil's-eye, Devil's Guts, Devil's Herb, Devil's Plague, Dew of the Sea, Dhup, Digitalis Purpurea, Di Huang, Dill, Dillisk, Dillseed, Dillweed, Dilly, Dilsk, Dilo Oil Tree, Dioscorea Villosa, Diosma Betulina, Dipsacus Sylvestris, Dipteryx Odorata, Divale, Djamboe, Doda, Dodan, Doadni, Dodder, Dog Brier, Dog Grass, Dog Rose, Dog's Mercury, Dog Tree, Dogwood, Dong Chong Xia Cao, Dong Quai, Dovefoot, Drago, Dragon's Blood, Dragon's mugwort, Dragonwort, Dropsy Plant, Drosera Rotundifolia, Dryopteris filix-mas, Ducks Foot, Dulse, Dutch Clover, Dwale, Dwarf Juniper, Dwarf Nasturtium, Dwayberry, Dyeberry, Dyer's Broom, Dyer's Greenweed, Dyer's Madder, Dyer's-saffron, Dysphania ambrosioides, Early Winter Cress, Earth-smoke, Easter Flower, Easter Giant, Eau-de-cologne Mint, Echinacea, Echinacea Angustifolia, Egg Fruit, Egg Wrack, Egyptian Privet, Eight-horned Anise, Eight-horns, Ela, Elaci, Elder, Elder-berry, Elder-flower, Elecampane, Eletteria Cardamomum, Eleuthero, Eleutherococcus Senticosus, Elk Mint, Emetic Herb, Enandi, Endive, English Alder, English Balm, English Catnip, English Chamomile, English Hawthorn, English Holly, English Hop, English Mandrake, English Serpentary, English Thyme, English Valerian, English Violet, English Wallflower, Epazote, Ephedra, Ephedra Nevadensis, Ephedra Sinica, Epifagus virginiana, Epilobium Angustifolium, Epimedium, Epimedium grandiflorum, Equisetum Arvense, Erigeron canadensis, Eriodictyon Californicum, Eruca vesicaria sativa, Erythraea Centaurium, Erythroxylum Catuaba, Erythroxylum Coca, Eschscholzia Californica, Espinheira Santa, Estragon, Ethiopian Cumin, Eucalyptus, Eucalyptus Globulus, Eugenia Carophyllata, Eupatorium, Eupatorium Perfoliatum, Eupatorium Purpureum, Euphorbia Hirta, Euphrasia Officinalis, European Alder, European Angelica, European Ash, European Barberry, European Black Alder, European Buckthorn, European Centaury, European Chestnut, European Cowslip, European Dill, European Elder, European Holly, European Hop, European White Water lily, European Willow, Euterpe Oleracea, Evening Primrose, Evening Star, Evergreen, Eye Balm, Eyebright, Eyeroot, Fagopyrum Esculentum, Fagus Grandifalia, Fah Tolai, Fairy Cup, Fairy's Glove, False Acacia, False Box, False Chamomile, False Jasmine, False Saffron, False Unicorn, False Valerian, False White Cedar, Featherfew, Featherfoil, Feather Geranium, Febrifuge Plant, Felon Herb, Felonwort, Female Fern, Fennel, Fenugreek, Ferula Asafoetida, Fetid Horehound, Fever Bush, Feverfew, Fever Grass, Fever Tree, Feverwort, Fiber, Ficus religiosa, Field Balm, Field Pansy, Field Poppy, Field Pumpkin, Field Sorrel, Figwort, Filipendula Ulmaria, Fir, Fir Balsam, Fireweed, Fir Pine, Fishfuddle, Five-fingers, Five-leaf, Flag Lily, Flagroot, Flanders Poppy, Flannelleaf, Flat-leaved Parsley, Flax, Flax Seed, Flax Weed, Fleabane, Flea Seed, Flesh and Blood, Fleur-de-lis, Flinders Rose, Florentine Iris, Florida Dogwood, Florida Fishpoison Tree, Flower de-luce, Flowering Dogwood, Flowering Willow, Flowery Knotweed, Foeniculum Vulgare, Folk's Glove, Food Of The Gods, Forsythia, Forsythia suspensa, Fo Ti, Foxberry, Fox Geranium, Foxglove, Fox Grape, Foxtail, Fragaria ananassa, Fragaria Vesca, Fragrant Balm, Fragrant Giant Hyssop, Fragrant Sumac, Frankincense, Fraxinus Americana, Fraxinus Excelsior, French Basil, French Lilac, French Parsley, French Rose, French Sorrel, French Tarragon, French Thyme, Friar's Cap, Fringe Tree, Fritillaria, Fritillaria Thunbergii, Frog Plant, Fucus Vesiculosus, Fu-ling, Fuller's-herb, Fumaria officinalis, Fumitory, Gagroot, Galangal, Galega Officinalis, Galipea officinalis, Galium Aparine, Galium Odoratum, Galium Verum, Gambooge, Gan cao, Gandana, Ganja, Gan-j iang, Ganoderma Lucidum, Gao Liang, Garabato, Garcinia, Garcinia cambogia, Garcinia gummi-gutta, Garcinia Kola, Garden Angelica, Garden Balm, Garden Basil, Garden Burnet, Garden Chamomile, Garden Chervil, Garden Chicory, Garden Dill, Garden Heliotrope, Garden Hyssop, Garden Lavender, Garden Loosestrife, Garden Marigold, Garden Mint, Garden Myrrh, Garden or Green Purslane, Garden Patience, Garden Rosemary, Garden Rue, Garden Sage, Garden Thyme, Garden Violet, Garlic, Garlic Chives, Garlic Sage, Gaultheria Procumbens, Ge-gen, Gelsemium Sempervirens, Genista, Genista Tinctoria, Gentian, Gentiana Lutea, Geranium Maculatum, Geranium Robertianum, Geraniums, German Chamomile, Germander, German Mustard, German Rue, German Tarragon, German Thyme, German Valerian, Geum urbanum, Ghaap, Gill Run Over, Ginger, Ginkgo, Ginkgo Biloba, Ginkgo Nut, Ginny Grains, Ginny Papper, Ginseng, Glechoma Hederacea, Glossy Buckthorn, Glycine max, Glycyrrhiza Glabra, Goathead, Goat's Rue, Goatweed, Goat Wort, Gold Coin Grass, Golden Aspen, Goldenberry, Golden Flower of Mary, Golden Loosestrife, Golden Ragwort, Golden Root, Goldenrod, Goldenseal, Golden Senecio, Gold Melissa, Goldy Star, Goosefoot, Goose Grass, Goosewort, Gorikapuli, Gospel Tree, Gossypium Hirsutum, Gotu Kola, Gourmet Parsley, Goutweed, Gow Choy, Graines, Grains Of Paradise, Gramineus, Grape, Grape Vine, Grass, Grass Burdock, Gravelroot, Graviola, Graybeard, Greasewood, Great Burdock, Greater Burnet, Greater Cardamom, Great Morel, Great Nettle, Great Stinging Nettle, Great Wild Valerian, Greek Hayseed, Green Ginger, Green Ozier, Green Tea, Grifola Frondosa, Grindelia, Grindelia Camporum, Groats, Ground Berry, Ground Cherry, Ground Holly, Ground Ivy, Ground Juniper, Ground Lemon, Ground Lily, Groundnut, Ground Pine, Ground Raspberry, Grouse Berry, Guaiac, Guaiacum, Guajacum, Guaiacum Officinale, Gualtheria Procumbens, Guarana, Guasai, Guava Tree, Guelder Rose, Guggul, Gui, Guinea Grains, Guinea Pepper, Guinea Seeds, Gum Bush, Gum Guggulu, Gum Myrrh Tree, Gumplant, Gurmabooti, Gurmar, Gymnema, Gymnema sylvestre, Gynostemma, Gynostemma pentaphyllum, Gypsyweed, Gypsywort, Habanero Pepper, Hackmatack, Hai-ts'ao, Hamamelis Virginiana, Handflower, Happy Tree, Hapusha, Hardock, Hareburr, Hare's Ear Root, Harpagophytum Procumbens, Hartshorn Plant, Harvest Lice, Hasabis, Hashish, Hatomugi, Haw, Hawaii Nut, Hawkweed, Hawthorn, Haymaids, Hazelnut Tree, Heal-All, Heart Of The Earth, Heartsease, Heather, Hebanthe Paniculata, Hedge Bind Weed, Hedge Fumitory, Hedge Maid, Hediondilla, Helianthus Annuus, Heliotrope, Hellweed, Helmet Flower, Helonias Root, Hemlock, Hemp, Henbane, Henna, Herb Bennet, Herb-of-Grace, Herb of St. Barbara, Herb of The Angels, Herb-of-the-cross, Herb Robert, Hercules Woundwort, He Shou Wu, Hibiscus, Hibiscus Sabdariffa, Hieracium Pilosella, Hierochloe odorata, High Angelica, High Mallow, Hill Berry, Hina, Hind Heal, Hineheel, Hing, Hini, Hippophae rhamnoides, Hip Rose, Hoarhound, Hock-heal, Hodoimo, Hoelen, Hog Apple, Hog Bean, Hog Cranberry, Hogweed, Holigold, Holly, Holy Basil, Holy Ghost Plant, Holy Herb, Holy Grass, Holy Thistle, Honey Plant, Honeysuckle, Hoodia, Hoodia pilifera, Hood Weed, Hoodwort, Hook-heal, Hopniss, Hops, Hops Vine, Horehound, Horny Goatweed, Horse Balm, Horse Chestnut, Horsefly Weed, Horseheal, Horse Mint, Horseradish, Horse Savin, Horsetail, Horse Thistle, Horseweed, Ho She Wu, Ho Shou Wu, Hot Mint, Hsia-ku-ts'ao, Hsiao-hui-hsiang, Hsieh-tzu-ts'ao, Hua-Hsian, Huang Qi, Huang Quin, Huarango, Hu-chin-ts'ao, Huckleberry, Hu-lu-ba, Hu-lu-pa, Humulus Lupulus, Hungarian Chamomile, Huo Ma Ren, Hurrburr, Hurtleberry, Hurtsickle, Husk Cherry, Hydnocarpus, Hydnocarpus kurzii, Hydrangea, Hydrangea Arborescens, Hydrastis Canadensis, Hyoscyamus Niger, Hypericum, Hypericum Perforatum, Hyssop, Hyssopus Officinalis, Iceland Lichen, Iceland Moss, I-chi-kao, Ignatia Amara, Ignatius Bean, Ilang-Ilang, Ilex Aquifolium, Ilex Paraguariensis, Illicium verum, Ill-scented Sumac, Imburana De Cheiro, Incensier, Indian Apple, Indian Arrowroot, Indian Balm, Indian Balmony, Indian Bedellium, Indian Borage, Indian Bread, Indian Chickweed, Indian Corn, Indian Dye, Indian Elm, Indian Gentian, Indian Ginseng, Indian Gooseberry, Indian Lotus, Indian Mustard, Indian Nettle, Indian Nut, Indian Olibanum, Indian Paint, Indian Pennywort, Indian Pink, Indian Plant, Indian Plume, Indian Potato, Indian Red Paint, Indian Root, Indian Sage, Indian Shamrock, Indian Snakeroot, Indian Tobacco, Indian Tree, Indigo Broom, Inonotus obliquus, Inula Helenium, Ipecac, Ipecac Shrub, Ipio, Iporoni, Iporuru, Iris, Iris Florentina, Iris Germanica, Irish Broom, Iris pallida, Irish Moss, Iris Versicolor, Italian Burnet, Italian Cress, Italian Jasmine, Italian Lovage, Italian Pimpernel, Ivory Plum, Jaborandi, Jackfruit, Jack Tree, Jak, Jacob's Chariot, Jacob's-staff, Jacon, Jamaican Dogwood, Jamaica Pepper, Jamaica Sorrel, Jambu, Jambul, Jamestown Weed, Japanese Catnip, Japanese Grapefruit, Japanese Horseradish, Japanese Mint, Japanese Mushroom, Japanese Seaweed, Jasmin, Jasmine, Jasmini Flos, Jasminum spp., Jateorhiza Palmata, Jaundice Berry, Jaundice Root, Java Pepper, Java Plum, Jersey Tea, Jerusalem Oak, Jessamine, Jesuit's Balsam, Jesuit Tea, Jew's-harp Plant, Jiaogulan, Jicara, Jimsonweed, Jing Jie, Jin Qian Cao, Jin Yin Hua, Job's Tears, Joe Pye Weed, Juglans cinerea, Juglans Nigra, Juglans Regia, Johnny-jump-up, Johnswort, Joint Fir, Ju Hua, Juniper, Juniper Bark, Juniper Berry, Juniper Bush, Jupiter's Bean, Juniperus Communis, Kachur, Kalmegh, Kamoteng Kahoy, Kanma, Kan-ts'ao, Kappa, Katphala, Kava Kava, Kelp, Kelpware, Kemangen, Keyflower, Key of Heaven, Khas Khas, Khat, Khella, Kiawe, Kidney Stone Tree, King Of Bitters, King's-clover, King's Crown, King's Cure, King's-cure-all, Kinnikinnick, Kiryat, Klamath Weed, Knitback, Knitbone, Knotgrass, Knotted Kelp, Knotted Marjoram, Knotted Wrack, Knotty Brake, Knotweed, Kola Nut, Korean Ginseng, Kua-lou, Kuawa, Kudzu, Kuei, Kumari, Kumaru, Kuo-lao, K'u-tou, Ku Ts'ai, Lactucarium, Lactuca Virosa, Ladder-to-heaven, Ladies'-delight, Ladies' Seal, Lady Bleeding, Lady's Bedstraw, Lady's Slipper, Lady's Mantle, Lady's-washbowl, Lai-ei-ts'ao, Lamb Mint, Lamium Album, Lammint, Lang-tu, Langue de Boeuf, Lapacho, Lappa, Lappa Minor, Large Fennel, Large-leaved Germander, Larrea Tridentata, Latherwort, Laurus Nobilis, Lavender, Lavender Giant Hyssop, Lavandula officinalis, Lawn Chamomile, Lawsonia inermis, Leafcup, Lebanese Oregano, Leeks, Lemon, Lemon Balm, Lemon Thyme, Lemon Verbena, Lemongrass, Lentinus Edodes, Leonurus Cardiaca, Leopard's Bane, Lepidium meyenii, Leptandra, Leptandra Virginica, Leptospermum scoparium, Lesser Indian Cress, Lesser Periwinkle, Le-ts'ao, Lettuce Opium, Levisticum Officinale, Lian Qiao, Licorice, Licorice Mint, Licorice Root, Life Root, Lignum Vitae, Ligusticum Porteri, Ligustrum Vulgare, Lily Convalle, Lily of the Valley, Limaosinho, Limeblossom, Lime Flowers, Lime Mint, Lime Tree, Linden, Linden Flower, Lindera benzoin, Ling Chi, Ling-t'ung, Ling Zhi, Link, Linseed, Lint Bells, Linum Usitatissimum, Lion's Ear, Lion's Foot, Lion's Tail, Lion's Tart, Lion's Tooth, Lippia graveolens, Lipstick Tree, Liquorice, Live-Forever, Live-Long, Liver Lily, Liverwort, Lizard's Tail, Lobelia, Lobelia Inflata, Longevity Herb, Lonicera Caprifolium, Lonicera Japonica, Lonicera Spp, Loodroot, Loosestrife, Lophophora williamsii, Lotus, Lotus Corniculatus, Lousewort, Lovage, Love Apples, Love in Winter, Love-lies-bleeding, Love Persley, Love Vine, Lucerne, Lu Hui, Lungwort, Luole, Lychee, Litchi chinensis, Lycium, Lycium Chinense, Lycium Fruit, Lycopodium, Lycopodium clavatum, Lycopus americanus, Lysimachia christinae, Lysimachia vulgaris, Lythrum salicaria, Ma Bian Cao, Macadamia Nut, Macadamia spp., Maca, Maca-Maca, Mace, Macochihua, Madagascar Periwinkle, Mad Apple, Madder, Madder Root, Madderwort, Mad Dog, Madweed, Madrone Tree, Ma huang, Maidenhair Fern, Maidenhair Tree, Maid's-hair, Maino, Maitake, Maize, Maka, Malabar Cardamom, Malabar Plum, Malabar Tamarind, Mal-dos-sete-dias, Male Fern, Mallow, Malpighia species, Malus Communis, Malva Sylvestris, Mamey Sapote, Manac, Mandioc, Mandragora, Mandragora Officinarum, Mandrake, Mangosteen Oil Tree, Manihot esculenta, Manioc, Manioc Root, Manna Grass, Man-t'ien-hsing, Manuka, Manuka Myrtle, Manuka Tree, Manzanilla, Maqui, Maramar, Maranta Arundinaceae, Maranta Starch, Marapuama, Mare's Tail, Marigold, Maroochi Nut, Marrubium, Marrubium Vulgare, Marshmallow, Marsh Marigold, Marsh Parsley, Marsh Trefoil, Marsh Woundwort, Marum, Marvel, Mary Bud, Mary Golde, Mary Gowles, Mary Jane, Maryland Pink, Mary's Grass, Mary's Mantle, Mary's Thistle, Master of the Woods, Masterwort, Mastic, Mate, Matricaria Chamomilla, May, Mayapple, May Blossom, Maybush, May Lily, Maypop, Maytenus, Maytenus Species, May Tree, Meadow Clover, Meadow Eyebright, Meadow Saffron, Meadow Sage, Meadowsweet, Mealberry, Medicago Sativa, Mei-ts'ao, Melaleuca, Melaleuca Alternifolia, Melegueta Pepper, Melilot, Melilotus Officinalis, Melissa, Melissa Officinalis, Melmot Berry, Mentha haplocalyx, Mentha piperita, Mentha pulegium, Mentha requienii, Mentha suaveolens, Mentha spicata, Menthol Mint, Mentha×piperita citrata, Menyanthes Trifoliata, Mercurialis Perennis, Mescal, Meshasringi, Mesquite, Methi, Mexican Mint, Mexican Oregano, Mexican Poppy, Mexican Potato, Mexican Tea, Mexican Thyme, Mexican Wild Yam, Mexico Seed, Middle Comfrey, Mignonette Tree, Mi-kan, Milfoil, Milk Ipecac, Milk Thistle, Milkwort, Millefoil, Mint, Mints, Miracle Herb, Miracle Tea, Mistletoe, Mitchella Repens, Mi-tieh-hsiang, Mi-ts'ao, Moccasin Flower, Mogo, Molina, Mo Li Hua, Momordica Charantia, Monarda Didyma, Monkey-Bread Tree, Monkshood, Monk's Pepper, Moonflower, Moon Grass, Moose Elm, Morinda, Morinda citrifolia, Morinda officinalis, Mormon Tea, Moroccan Ironwood, Mortification Root, Moms Nigra, Mother-of-thyme, Mother's-heart, Motherwort, Moujean Tea, Mountain Arnica, Mountain Ash, Mountain Aspen, Mountain Balm, Mountain Berry, Mountain Box, Mountain Cranberry, Mountain Daisy, Mountain Grape, Mountain Holly, Mountain Mint, Mountain Savory, Mountain Strawberry, Mountain Tobacco, Mountain Tea, Mouse-ear, Mugwort, Muira Puama, Mujonso, Mulberry, Mullein, Murraya koenigii, Muscatel Sage, Mu-Su, Mu Tong, Mu-yao, Myrciaria dubia, Myrica, Myrica Cerifera, Myricae Cortex, Myristica Fragrans, Myroxylon Balsamum, Myroxylon Pereirae, Myrrh, Myrrhis odorata, Myrtle, Myrtle Pepper, Myrtus communis, Nagara, Naidi, Naked Ladies, Napa Cabbage, Nappa, Narrow Dock, Narthex, Nashia inaguensis, Nasturtium Officinale, Naughty Man's Cherries, Neem, Nelumbo nucifera, Nenuphar, Nep, Nepeta Cataria, Nerveroot, Nettle, Nettle Flowers, New England Pine, New Jersey Tea, New Zealand Tea Tree, Niando, Nicotiana Rustica, Nightshade, Night Willow Herb, Nine Hooks, Nine Joints, Nip, Nira, Niu Bang, Noble Chamomile, Noble Yarrow, Nodding Wakerobin, Noni, Normandy Cress, Northern Pine, Northern Spicebush, Northern White Cedar, Norwegian Kelp, Nosebleed, Nutmeg, Nux Vomica, Nymphaea Alba, Oak, Oats, Ocimum basilicum, Ocimum tenuiflorum, Oenothera Biennis, Ohio Curcuma, Oil plant, Oilnut, Oil Nut Tree, Old English Lovage, Old-maid's-nightcap, Old-maid's-pink, Old Man, Old-man's-beard, Old Man's Nightcap, Old Man's Pepper, Old woman, Olea Europaea, Olibanum, Olive, Omam, Omum, One-berry, Onion, Opium Poppy, Oplopanax horridus, Orange, Orange Mint, Orange Root, Ordeal Bean, Oregano, Oregano Brujo, Oregon Grape, Oriental Garlic, Oriental Mustard, Oriental Poppy, Origanum majorana, Origanum syriacum, Origanum vulgare, Orpine, Orris Root, Osha, Osterick, Oswego Tea, Our-Lady's-bedstraw, Our-Lady's-tears, Oval Buchu, Owler, Oxadoddy, Ox Balm, Ox Heart, Ox-tongue, Pacific Madrone, Pacific Yew, Padmaka, Paeonia Officinalis, Paico, Paigle, Palisade Pine, Palma Christi, Palmaria Palmata, Palmetto, Panax Ginseng, Panax Quinquefolium, Panay, Pansy, Papaver orientale, Papaver Rhoeas, Papaver Somniferum, Papaya, Paper Birch, Papoose Root, Paracress, Paraguay Tea, Pareira, Parell, Parietaria Officinalis, Pariswort, Parsley, Parsnip, Partridgeberry, Pasque Flower, Passiflora, Passiflora Incarnata, Passion Flower, Passions, Passion Vine, Pastinaca sativa, Patchouli, Patience Dock, Patience Herb, Pau d'Arco, Paullinia Cupana, Pausinystalia Yohimbe, Pauson, Peepal Tree, Pei-ma, Peking Cabbage, Pelargonium, Pelargonium sidoides, Pelargonium species, Pellitory, Pellitory Of The Wall, Pembina, Pennyroyal, Peony, Pepperidge Bush, Peppermint, Pepperweed, Perfume Tree, Periploca Of The Woods, Persea Americana, Persian Berries, Persicaria odorata, Personata, Peruvian Balsam, Peruvian Bark, Peruvian Ginseng, Peruvian Ground Cherry, Petasites Hybridus, Petokal, Petroselinum Crispum, Peumus Boldus, Peyote, Pfaffia paniculata, Philanthropos, Phudina, Phyllanthus, Phyllanthus emblica, Phyllanthus niruri, Physalis Alkekengi, Physalis Peruviana L., Physic Root, Physostigma venenosum, Phytolacca Americana, Picrasma Excelsa, Pigeon's Grass, Pignoli, Pigweed, Pikake, Pill-Bearing Spurge, Pilocarpus Microphyllus, Pilosella, Pimbina, Pimenta Dioica, Pimenta Officinalis, Pimpinella Anisum, Pine, Pine Nut, Pineapple, Pineapple Strawberry, Pineapple Verbena, Pinkroot, Pink Rose, Pinon Nut, Pinus spp., Pinus Strobus, Pinus Sylvestris, Pinyon Pinenut, Piper betle, Piper cubeba, Piper Methysticum, Piper Nigrum, Pipe Tree, Pipsissewa, Pistachio, Pistacia vera, Piscidia piscipula, Pissabed, Pistachio, Plantago Major, Plantago Psyllium, Plantago Seed, Plantain, Plectranthus amboinicus, Pleurisy Root, Plum Rose, Podophyllum Peltatum, Poet's Jasmine, Pogostemon cablin, Poha Berry, Poison Ash, Poison Flag, Poison Parsley, Poison Tobacco, Pokeroot, Pokeweed, Polar Plant, Polygala Senega, Polygonatum Multiflorum, Polygonum Aviculare, Polygonum Bistorta, Polygonum Multiflorum, Polygonum odoratum, Polypodium Vulgare, Polypody, Poor Man's Ginseng, Poor-man's-treacle, Poplar, Popotillo, Populus alba, Populus Tremuloides, Poria, Poria cocos, Portulaca Oleracea, Pot, Potato Bean, Potency Wood, Potentilla Anserina, Potentilla Erecta, Potentilla Reptans, Potenzholz, Pot Marigold, Pouteria sapota, Pouteria campechiana, Prairie Smoke, Prickly Ash, Prickly Lettuce, Prickly Pear Cactus, Prickly Poppy, Priest's-crown, Prim, Primrose, Primula Veris, Prince's Feather, Prince's-pine, Privet, Prosopis pallida syn. Prosopis limensis, Provence Rose, Prunella, Prunella Vulgaris, Prunus Amygdalus, Prunus Dulcis, Prunus Serotina, Prunus Spinosa, Psidium Guajava, Psoralea, Psoralea corylifolia, Psoralea Fruit, Psyllium, Ptychopetalum ovata, Pueraria Lobata, Puff Ball, Pu gong ying, Pukeweed, Pu-kung-ying, Pulmonaria Officinalis, Pulsatilla, Pumpkin, Pumpkin Pine, Puncture Vine, Purging Buckthorn, Purple Angelica, Purple Betony, Purple Clover, Purple Coneflower, Purple Foxglove, Purple Leptandra, Purple Loosestrife, Purple Medic, Purple Passionflower, Purple Rocket, Purplestem Angelica, Purslane, Pygeum, Pygeum Africanum, Pyrethrum, Pyrola Umbellata, Quack Grass, Quaking Aspen, Quassia, Quassia Bark, Quassia Wood, Quebra Pedra, Queen Annes Lace, Queen Of The Meadow, Queensland Nut, Queen's Delight, Queen's-root, Quercus alba, Queue de Lezard, Quickbeam, Quick-set, Quince, Quinine Tree, Quinsy Berries, Quiverleaf, Race Ginger, Racoon Berry, Rainbowweed, Rashona, Raspberry, Raspberry Leaf, Rat's Tail, Rattlebush, Rattleroot, Rattlesnake Root, Rattleweed, Rau Ram, Rauwolfia, Rauwolfia Serpentina, Red Alder, Red Balm, Red Bearberry, Red-bearded, Red Bergamot, Redberry Tea, Red Bush Tea, Red Clover, Redcole, Red Dulse, Red Eyebright, Red Legs, Red Paint Root, Red Pollom, Red Poppy, Red Puccoon, Red Raspberry, Red Robin, Red Root, Red Root Sage, Red Rose, Red Sage, Red Sorrel, Red Sunflower, Red Tea, Red Trillium, Red-veined Dock, Reefer, Rehmannia, Rehmannia Glutinosa, Reishi, Rhamnus Cathartica, Rhamnus Frangula, Rhamnus Purshiana, Rheumatism Root, Rheumatism Weed, Rheum Palmatum, Rhodiola, Rhodiola sacra, Rhubarb, Rhus trilobata, Ribes Nigrum, Ribwort, Richweed, Ricinus Communis, Rimed scutatus, Ritha, Robinia Pseudoacacia, Rock Brake, Rock Fern, Rockweed, Roman Chamomile, Roman Cumin, Roman Fennel, Rooibos, Root Of The Holy Ghost, Roquette, Rosa Canina, Rosa Centifolia, Rosa Gallica, Rose, Rose Apple, Roselle, Rosemary, Rosemary Plant, Rose-noble, Rose Root, Rosin Rose, Rosmarinus Officinalis, Rosy Periwinkle, Rou Dou Kou, Round Buchu, Round-leaved Dock, Round-leafed Mint, Round-leaved Sorrel, Rowan Tree, Royal Herb, Royal Jasmine, Rub Cherry, Rubia tinctoria, Rubus Fructicosus, Rubus Idaeus, Rucola, Rue, Rugula, Rumara, Rumex Acetosella, Rumex Crispus, Rumex Hymenosepalus, Rumex Obtusifolius, Running Club Moss, Ruscus Aculeatus, Russian Chamomile, Russian Mustard, Rustic's Treacle, Ruta Graveolens, Sabal, Saccharum officinarum, Sacred Bark, Sacred Basil, Sacred Fig, Sacred Lotus, Sacred Plant, Sacred Sage, Sacred Tree, Sacred Water Lotus, Safflower, Saffron, Sagackhomi, Sage, Sage-leaved Germander, Sage Of Bethlehem, Sakè, Salad Burnet, Salad Chervil, Salad Rocket, Salai Gugal, Salix Alba, Saloip, Salvia, Salvia apiana, Salvia hispanica, Salvia Miltiorrhiza, Salvia Officinalis, Salvia Sclarea, Sambucus Nigra, Sampson Root, Sandalwood, Sandberry, Sangre de Drago, Sangue de Drago, Sanguinaria, Sanguinaria Canadensis, Sanguisorba Minor, Sanguisorba Officinalis, Sanicle, Sanicula Europaea, Sanicula Marilandica, Santalum Album, Sapin, Sapindus mukorossi, Saponaria Officinalis, Sapote, Sarapia, Sarepta Mustard, Sarothamnus Scoparius, Sarpagandha, Sarsaparilla, Sassafras, Sassafras Albidum, Satan's Apple, Satavar, Satinflower, Satureja hortensis, Satureja montana, Saventaro, Savory, Saw Palmetto, Saxifrax, Scabish, Scabwort, Scarlet Bergamot, Scarlet Monarda, Scarlet Sage, Scarweed, Scented Fern, Scented Sumac, Schisandra, Schisandra Chinensis, Schizonepeta, Schizonepeta tenuifolia, Scopolia, Scopolia carniolica, Scotch Broom, Scotch Fir, Scotch Heather, Scotch Pine, Scots Pine, Scouring Rush, Scrofula Plant, Scrophularia Nodosa, Scurfy Pea, Scurvy Grass, Scurvy Weed, Scutellaria Baicalensis, Scutellaria Lateriflora, Sea Buckthorn, Sea Lettuce Flakes, Sea Oak, Sea Onion, Sea Parsley, Seaweed, Seawrack, Sedum Telephium, Self-Heal, Seneca Snakeroot, Senecio Aureus, Seneca Grass, Senega, Senega Root, Senna, Sereh, Serenoa Repens, Serpentary, Serpyllum, Setwall, Shameface, Shan-cha, Sha-ren, Shan-yao, Shatamull, Shatapushpa, Shatavari, Shave Grass, Shea Tree, Sheepberry, Sheep Sorrel, Sheng Di, Sheng Di Huang, Sheng Ti Huang, Shen Jiang, Shepherd's Knot, Shepherd's Needle, Shepherd's Purse, Shi Chang Pu, Shield Fern, Shih-lo, Shihuahuaco, Shiitake, Shiny Asparagus, Shirokikurage, Shoofly, Short Buchu, Short-leaved Buchu, Shovelweed, Shu Di Huang, Shu Ti Huang, Siberian Ginseng, Sicklewort, Silkweed, Silver Birch, Silver Fir, Silver Leaf, Silver-leaf Poplar, Silver Mint, Silver Pine, Silver Poplar, Silver Tree-ear Fungus, Silverweed, Silybum, Silymarin, Simply Jack, Sinapis alba, Skullcap, Skunkbush, Skunkbush Sumac, Skunk Cabbage, Slippery Elm, Sloe, Smallage, Smallanthus sonchifolius, Small Nasturtium, Smelling-stick, Smilax Uti Lis, Smooth Cicely, Smooth Strophanthus, Snakebite, Snake Lily, Snake Root, Snakeweed, Snapping Hazelnut, Snap-Wood, Snowball Tree, Snowdrop Tree, Snowflake, Snowflower, Snow Fungus, Soap Berry, Soapnut, Soapwort, Soft Pine, Solanum Dulcamara, Soldier's Cap, Soldier's Woundwort, Solidago Canadensis, Solidago Virgaurea, Solis Sponsa, Solomon's Seal, Solsequia, Son-before-the-father, Sorbus Aucuparia, Sour Dock, Sour Grass, Soursop, Sour Weed, Southern Ginseng, Southernwood, Sowberry, Soy, Soya, Soybean, Spanish Chamomile, Spanish Chestnut, Spanish Jasmine, Spanish Thyme, Spearmint, Speedwell, Spiceberry, Spicebush, Spicewood, Spicy Wintergreen, Spigelia Marilandica, Spike Lavender, Spiked Loosestrife, Spikenard, Spilanthes acmella, Spoonwood, Spoonwort, Spotted Alder, Spotted Thistle, Spring Cress, Spring Wintergreen, Square Stalk, Squawbush, Squaw Root, Squaw Tea, Squaw Vine, Squill, Stachys Officinalis, Stachys Palustris, Stagbush, Staghorn, Stanchgrass, Star Anise, Starbloom, Star Flower, Star Fruit, Star Grass, Starweed, Starwort, Stellaria, Stellaria Media, Stickwort, St. Ignatius Bean, Stillingia Sylvatica, Stingnose, Stinking Benjamin, Stinking Christopher, Stinging Nettle, Stingless Nettle, Stinking Nightshade, Stinking Roger, Stinking Rose, Stinking Weed, Stinking Willie, Stinkweed, Stitchwort, St. John's Bread, St. John's Grass, St. John's Plant, St. John's Wort, St. Josephwort, Stonecrop, Stone Root, Strawberry, Stork's Bill, Strawberry Tomato, Strawberry Tree, Striped Alder, Strophanthus, Strophanthus Gratus, Strychnine, Strychnine Tree, Strychnos nux-vomica, Styrax Benzoin, Succory, Sudanese Tea, Sugar Cane, Sui Hoi, Suma, Su Nanesi, Sundew, Sunflower, Sunkfield, Sunthi, Surasa, Suterberry, Swallowwort, Swamp Cedar, Swamp Root, Sweating Plant, Sweet Almond, Sweet Balm, Sweet Basil, Sweet Bay, Sweet Birch, Sweet Bracken, Sweet Brake, Sweet Bugle, Sweet Cane, Sweet Chervil, Sweet Chestnut, Sweet Cicely, Sweet Clover, Sweet Coltsfoot, Sweet Cumin, Sweet Dock, Sweet Elm, Sweet Fennel, Sweet Fern, Sweet Flag, Sweet Flag Rhizome, Sweet Goldenrod, Sweet Grass, Sweet Iris, Sweet Lavender, Sweet Licorice, Sweet Lucerne, Sweet Marjoram, Sweet Myrrh, Sweet Root, Sweet Rush, Sweet-scented Geranium, Sweet Tea Vine, Sweet Tongue, Sweet Violet, Sweetweed, Sweet Wood, Sweet Woodruff, Swertia, Swertia chirayita, Swine Snout, Symphytum Officinale, Symplocarpus Foetidus, Syrian Oregano, Syzygium cumini, Syzygium jambos, Tabebuia Spp., Tagara, Taheebo, Ta-huang, Tailed Cubebs, Tailed Pepper, Tailwort, Tall Nasturtium, Tallow Shrub, Tall Speedwell, Tall Veronica, Tamanu Nut Tree, Tamarind, Tamarindus Indica, Tamus, Tanacetum Parthenium, Tanacetum Vulgare, Tang Kuei, Tanners Bark, Tanner's-dock, Tan Shen, Tansy, Tap Aloe, Tapioca-root, Taraxacum Officinale, Tarragon, Tartar Root, Tarweed, Taxus Brevifolia, Teaberry, Teasel, Tea Tree, Te Limon, Telltime, Tenuifolia, Terminalia Arjuna, Tetterberry, Tetterwort, Teucrium marum, Teucrium scorodonia, Thali, Theobroma Cacao, Thorn Apple, Thorn Poppy, Thorntree, Thorny Burr, Thoroughwort, Thousand-leaf, Thousand-seal, Three-leaved Caper, Three-leaved Nightshade, Throatwort, Thuja, Thuja Occidentalis, Thumb, Thunberg Fritillaria Bulb, Thyme, Thymus citriodorus, Thymus Serpyllum, Thymus Vulgaris, Tian Men Dong, Tibetan Rhodiola, T'ien-shih-li, Ti Huang, Ti Huang Chiu, Tilia Americana, Tilia Cordata, Tilia Europea, Tipton Weed, Toad Flax, Tobacco Wood, Tokal, Tomillo, Tom Thumb Nasturtium, Tongue Grass, Tonka, Tonka Bean, Tonka Bean Tree, Tonquin Bean, Toothache Plant, Toothache Tree, Trachyspermum ammi, Treacle Mustard, Tree Moss, Tree of Joy, Tree of Life, Tree's Dandruff, Trefoil, Trembling Aspen, Trembling Poplar, Tremella fuciformis, Tribulus, Tribulus terrestris, Tricolor Garlic, Trifolium pratense, Trifolium repens, Trigonella, Trigonella Foenum-Graecum, Trillium, Trillium Erectum, Triticum aestivum, Tropaeolum minus, Tormentil, True Angostura, True Chamomile, True Lavender, True Oregano, True Sage, True Taragon, True Unicorn Root, Tse-lan, Tuber Root, Tuckahoe, Tulasi, Tulsi, Tumeric Root, Tuna Cactus, Turkey Burrseed, Turkish Oregano, Turmeric, Turnera Diffusa, Turtlebloom, Turtlehead, Tussilago Farfara, Twak, Twinflower, Tzu-mo-lo, Uassi, Ulmus Rubra, Uma, Umbrella Plant, Umcka, Umckaloabo, Una De Gato, Uncaria Tomentosa, Undaria pinnatifida, Upland Cotton, Upland Cress, Upland Cranberry, Uppagi, Upside-Down Tree, Urginea Maritima, Urtica Dioica, Usnea, Usnea spp., Uva Ursi, Vaccinium Macrocarpon, Vaccinium Myrtillus, Valerian, Valeriana Officinalis, Vandalroot, Vanilla, Vanilla fragrans, Vanilla Grass, Vanilla planifolia, Varuna, Vegetable Antimony, Vegetable Marrow, Vegetable Sulfur, Vegetable Tallow, Vegetable Wax, Venus' Basin, Venus'-hair Fern, Verbascum Thapsus, Verbena, Verbena Officinalis, Vermont Valerian, Vernonia Amygdalina, Veronica Beccabunga, Veronica Officinalis, Vervain, Vetiver, Vetiveria zizanioides, Vetivert, Viburnum Opulus, Viburnum Prunifolium, Vietnamese Cilantro, Vietnamese Coriander, Vietnamese Mint, Vinca Minor, Vinca Rosea, Vine, Viola Odorata, Viola Tricolor, Violet-bloom, Virginia Bugleweed, Virginia Dogwood, Virginia Prune, Virginia Skullcap, Virginia Snakeroot, Virginia Water Horehound, Viscum Album, Visnaga, Vitellaria paradoxa, Vitex, Vitex Agnus-Castus, Vitis Vinifera, Vomitroot, Wachsgagl, Wakame, Wake Robin, Waldmeister, Wallflower, Walnut, Wasabi, Wasabia japonica, Wasei, Water Bugle, Watercress, Water Dragon, Water Flag, Water Horehound, Water Hyssop, Water Lily, Water Mint, Water Pimpernel, Water Shamrock, Water Thistle, Wattle, Wax Cluster, Wax Dolls, Wax Myrtle, Way Bennet, Weeping Ash, Weeping Forsythia, Wheatgrass, Whinberry, Whippoorwill's-shoe, Whistling Thorn, White Archangel, White Ash, White Birch, White Bird's-eye, White Bryony, White Cedar, White Ceremonial Sage, White Chamomile, White Clover, White Deadnettle, White Endive, White Flower De Luce, White Horehound, White Jelly-leaf, White Muer, White Lotus, White Mustard, White Nettle, White Pine, White Poplar, White Sage, White Tansy, Whitethorn, White Tree-ear, White Turmeric, White Walnut, White Willow, Whitten Tree, Whorlywort, Whortleberry, Whorts, Wild Allspice, Wild Angelica, Wild Black Cherry, Wild Brier, Wild Bryony, Wild Carrot, Wild Celery, Wild Chamomile, Wild Chicory, Wild Cotton, Wild Crocus, Wild Endive, Wild Fennel, Wild Geranium, Wild Hops, Wild Indigo, Wild Iris, Wild Lemon, Wild Lettuce, Wild Mandrake, Wild Marjoram, Wild Oats, Wild Opium, Wild Pansy, Wild Parsnip, Wild Passionflower, Wild Pieplant, Wild Rhubarb, Wild Rye, Wild Snakeroot, Wild Strawberries, Wild Succory, Wild Sunflower, Wild Sweetsop, Wild Tansy, Wild Teasel, Wild Thyme, Wild Tobacco, Wild Valerian, Wild Vine, Wild Yam, Willow, Willow Herb, Windflower, Wind Root, Wineberry, Winter Berry, Winterbloom, Winter Cherry, Winter Clover, Wintergreen, Winterlien, Winter Marjoram, Winter Savory, Winter Thyme, Winterweed, Witches'-moneybags, Witchgrass, Witch Hazel, Witch's Bells, Withania, Withania Somnifera, Wolfsbane, Wolf s Claw, Woman's Long Hair, Wood Betony, Woodbine, Wood Boneset, Wood Ear Fungus, Woodland Germander, Woodland Strawberry, Wood Licorice, Woodruff, Wood Sage, Wood Strawberry, Wood Turmeric, Wood Vine, Woodward, Woody Nightshade, Woolly Mint, Woolly Thistle, Worm Grass, Wormseed, Wormweed, Wormwood, Woundwort, Wu-pa-ho, Wu-wei-zi, Wycopy, Xiao-hue-xiang, Xi Shu, Xu Ku Cao, Yacon, Yacuma, Yang-Mei, Yape, Yarrow, Yasmin, Yasti Madhu, Yawroot, Yellow Bedstraw, Yellow Cedar, Yellow Dock, Yellow Eye, Yellow Ginseng, Yellow Indian Paint, Yellow Indian Shoe, Yellow Indigo, Yellow Jasmine, Yellow Jessamine, Yellow Lark's Heels, Yellow Locust, Yellow Loosestrife, Yellow Melilot, Yellow Mustard, Yellow Paint Root, Yellow Poppy, Yellow Puccoon, Yellow Rocket, Yellowroot, Yellow Thistle, Yellow Vine, Yerba, Yerba Buena, Yerba Mansa, Yerba Manza, Yerba Santa, Yin-hsing, Yin Yang Huo, Ylang Ylang Tree, Yohimbe, Yohimbine, Yucca, Yucca spp., Yueh-kuei, Yuma, Yu Mi Shu, Yuzu, Yuyu Chonta, Zaatar, Zacate Limon, Zanthoxylum Americanum, Zea Mays, Zedoary, or Zingiber Officinale.

Probiotics can also be included in cannabinoid compositions prepared according to the invention. Examples of suitable probiotics include, but are not limited to, Acinetobacter calcoaceticus, Arthrobacter agilis, Arthrobacter citreus, Arthrobacter globiformis, Arthrobacter luteus, Arthrobacter simplex, Azotobacter chroococcum, Azotobacter paspali, Azospirillum brasiliense, Azospirillum lipoferum, Bacillus ssp. (e.g., Bacillus brevis, Bacillus coagulans, Bacillus laterosporus, Bacillus marcerans, Bacillus pumilus, Bacillus polymyxa, Bacillus sphaericus, Bacillus subtilis), Bacteroides lipolyticum, Bacteroides succinogenes, Bifidobacterium ssp. (e.g., Bifidobacterium animalis lactis, Bifidobacterium bifidum, Bifidobacterium infantis, Bifidobacterium lactis, Bifidobacterium longum, Bifidobacterium animalis, Bifidobacterium breve), Brevibacterium lipolyticum, Brevibacterium stationis, Enterococcus faecium, Kurthia zopfii, Lactobacillus ssp. (e.g., Lactobacillus acidophilus, Lactobacillus brevis, Lactobacillus bulgaricus, Lactobacillus casei, Lactobacillus delbrueckii LE, Lactobacillus gasseri, Lactobacillus helveticus, Lactobacillus lacris, Lactobacillus paracasei, Lactobacillus plantarumtarum, Lactobacillus reuteri, Lactobacillus rhamnosus, Lactobacillus salivarius, Lactobacillus sporogenes), Myrothecium verrucaria, Pseudomonas calcis, Pseudomonas dentrificans, Pseudomonas fluorescens, Pseudomonas glathei, Phanerochaete chrysosporium, Saccharomyces boulardii, Saccharomyces cerevisiae, Streptococcus thermophilus, Streptomyces fradiae, Streptomyces cellulosae, Streptomyces griseoflavus, and combinations thereof.

Homeopathic remedies can also be included in cannabinoid compositions prepared according to the invention. Examples of suitable homeopathic remedies and indications that can be treated with homeopathic remedies include, but are not limited to, adrenocorticotropic hormone (ATCH), Abies Canadensis, Abies Nigra, Abrotanum, Absinthium, Acacia Arabica, Acalypha Indica, Acetaldehyde, Acetanilidum, Aceticum Acidum, Acetylsalicylicum Acidum, Achyranthis Calea, Aconite or Aconitum Nap, Aconitum Ferox, Aconitum Lycoctonum, Aconitum Napellus, Aconitum, Radix, Acorns Calamus or Calamus, Actaea Spicata Acrylate, Actaea Rac or Cimicifuga, Actaea Spic, Adamas, Adelheidsquelle, Adenosinum Cyclophosphoricum, Adeps Suillus, Adipose Tissue, Adonis Vernalis, Adrenal Cortex, Adrenal Gland, Adrenalinum or Epinephrine, Adrenocorticotrophin, Aesculinum, Aesculus Carnea, Flos, Aesculus Glabra, Aesculus Hippocastanum, Aesculus Hippocastanum Flos, Aethiops Antimonialis, Aethiops Mercurialis-Mineralis, Aethusa Cynapium, Agaricinum, Agaricus Campanulatus, Agaricus Campestris, Agaricus Citrinus, Agaricus Emeticus, Agaricus Muscarius, Agaricus Pantherinus, Agaricus Phalloides, Agaricus Procerus, Agaricus Semiglobatus, Agaricus Stercorarius, Agave Americana, Agave Tequilana, Agnus Castus, Agraphis Nutans, Agrimonia Eupatoria, Agrimonia Eupatoria, Flos, Agrimonia Odorata, Flos, Agrostemma Githago, Ailanthus Glandulosus, Aletris Farinosa, Alfalfa, Alisma Plantago, Allium Cepa, Allium Sativum, Alloxanum, Alnus Glutinosa, Alnus Serrulata, Aloe Socotrina, Alstonia Constricta, Alstonia Scholaris, Althaea Officinalis, Alumen or Alum, Alumina, Alumina Silicata, Aluminum Metallicum, Aluminum Muriaticum, Ambra Gri sea, Ambrosia Artemisiaefolia, Ammi Visnaga, Ammoniacum Gummi, Ammonium Aceticum, Ammonium Benzoicum, Ammonium Bromatum, Ammonium Carbonicum, Ammonium Causticum, Ammonium Citricum, Ammonium Iodatum, Ammonium Muriaticum, Ammonium Nitricum, Ammonium Phosphoricum, Ammonium Picricum, Ammonium Tartaricum, Ammonium Valerianicum, Ammonium Vanadium, Amorphophallus Rivieri, Ampelopsis Quinquefolia, Amygdala Amara, Amygdalae Amarae Aqua, Amygdalae Amarae Oleum, Amygdalus Persica, Amyl Nitrosum, Anacardium Occidentale, Anacardium Orientate, Anagallis Arvensis, Ananassa, Anas Barbariae Hepatis Et Cordis Extractum, Anatherum Muricatum, Anchusa Officinalis, Anemone Nemorosa, Anemopsis Californica, Anethum Graveolens, Angelica Archangelica, Angelica Atropurpurea, Angelica Sinensis, Radix, Angophora Lanceolata, Angustura Vera, Anhalonium Lewinii, Anilinum, Anilinum Sulphuricum, Anisum, Anthemis Nobilis, Anthemis Pyrethrum, Anthoxanthum Odoratum, Anthracinum (anthrax), Antimonium Arsenicicum, Antimonium Crudum, Antimonium Iodatum, Antimonium Muriaticum, Antimonium Oxydatum, Antimonium Sulphuratum Aureum, Antimonium Tartaricum, Antipyrinum, Apatite, Apiolum, Apis Mellifica, Apis Venenum Purum, Apium Graveolens, Apocynum Androsaemifolium, Apocynum Cannabinum, Apomorphinum, Apomorphinum Muriaticum, Aqua Marina, Aquilegia Vulgaris, Aralia Hispida, Aralia Quinquefolia, Aralia Racemosa, Aranea Diadema, Arbutinum, Arbutus Andrachne, Areca Catechu, Argemone Mexicana, Argentum Cyanatum, Argentum Iodatum, Argentum Metallicum, Argentum Muriaticum, Argentum Nitricum, Argentum Oxydatum, Argentum Phosphoricum, Aristolochia Clematitis, Aristolochia Milhomens, Aristolochia Serpentaria, Arnica Montana, Arnica Montana, Radix, Arsenicum Album, Arsenicum Bromatum, Arsenicum Iodatum, Arsenicum Metallicum, Arsenicum Sulphuratum Flavum, Arsenicum Sulphuratum Rubrum, Artemisia Vulgaris, Arum Dracontium, Arum Italicum, Arum Maculatum, Arum Triphyllum, Arundo Mauritanica, Asafoetida, Asarum Canadense, Asarum Europaeum, Asclepias Curassavica, Asclepias Incarnata, Asclepias Syriaca, Asclepias Tuberosa, Asclepias Vincetoxicum, Asclepias Vincetoxicum Folia, Asimina Triloba, Asparagus Officinalis, Asperula Odorata, Astacus Fluviatilis, Asterias Rubens, Astragalus Menziesii, Atropinum, Atropinum Sulphuricum, Aurum Bromatum, Aurum Iodatum, Aurum Met, Arum Mur, Aurum Muriaticum Kalinatum, Aurum Muriaticum Natronatum, Aurum Sulphuratum, Avena Sativa, Aviaire, Azadirachta Indica, Bacillinum of Burnet, Badiaga, Baja, Balsamum Peru, Baptisia Tinctoria, Barosma Cren, Baryta Acetica, Baryta Carbonica, Baryta Iodata, Baryta Muriatica, BCG, Belladonna, Belladonna, Radix, Bellis Perennis, Benzinum, Benzinum Dinitricum, Benzoicum Acidum, Benzoinum, Berberinum, Berberis Aquifolium, Berberis Vulgaris, Berberis Vulgaris, Fructus, Beryllium Metallicum, Beta Vulgaris, Betainum Muriaticum, Betula Pendula, Cortex, Betula Pendula, Folia, Bismuthum Metallicum, Bismuthum Oxydatum, Bismuthum Subnitricum, Bixa Oreliana, Blatta Americana, Blatta Orientalis, Boldo, Boletus Luridus, Boletus Satanas, Bombyx Processionea, Borago Officinalis, Borax, Boricum Acidum, Botulinum, Bovista, Brassica Napus, Bromelain, Bromium, Bromus Ramosus, Flos, Brucinum, Bryonia Alba, Bufo Rana, Bunias Orientalis, Buthus Australis, Butyricum Acidum, Buxus Sempervirens, Cacao, Cactus Grandiflorus, Cadmium Bromatum, Cadmium Iodatum, Cadmium Metallicum, Cadmium Muriaticum, Cadmium Sulphuratum, Cadmium Sulphuricum, Caffeinum, Cahinca, Cajuputum, Caladium Seguinum, Calcarea Acetica, Calcarea Arsenicica, Calcarea Carbonica, Calcarea Caustica, Calcarea Fluorica, Calcarea Hypochlorata, Calcarea Hypophosphorosa, Calcarea Iodata, Calcarea Lactica, Calcarea Muriatica, Calcarea Oxalica, Calcarea Phosphorica, Calcarea Picrata, Calcarea Silicata, Calcarea Sulphurica, Calendula Officinalis, Calluna Vulgaris, Flos, Calotropis Gigantea, Caltha Palustris, Camphora, Camphora Monobromata, Camphoricum Acidum, Canchalagua, Candida Albicans, Candida Parapsilosis, Canine Dapp, Cantharidinum, Cantharis, Capsicum, Capsicum Annuum, Carbo Animalis, Carbo Vegetabilis, Carbolicum Acidum, Carboneum, Carboneum Chloratum, Carboneum Hydrogenisatum, Carboneum Oxygenisatum, Carboneum Sulphuratum, Carcinosinum, Cardiospermum, Carduus Benedictus, Carduus Marianus, Carpinus Betulus, Flos, Cartilago Suis, Carum Carvi, Cascarilla, Cassada, Castanea Sativa, Flos, Castanea Vesca, Castor Equi, Castoreum, Catalpa Bignonioides, Caulophyllum Thalictroides, Causticum, Ceanothus Americanus, Cedron, Celtis Occidentalis, Cenchris Contortrix, Centaurea Tagana, Centaurium Umbellatum, Flos, Cephalanthus Occidentalis, Cerasus Virginiana, Ceratostigma Willmottianum, Flos, Cereus Bonplandii, Cereus Serpentinus, Cerium Oxalicum, Cetraria Islandica, Chamomilla, Cheiranthus Cheiri, Chelidonium Majus, Chelidonium Majus, Radix, Chelone Glabra, Chenopodii Glauci Aphis, Chenopodium Anthelminticum, Chenopodium Vulvaria, Chimaphila Maculata, Chimaphila Umbellata, Chininum Arsenicicum, Chininum Arsenicosum, Chininum Muriaticum, Chininum Purum, Chininum Salicylicum, Chininum Sulphuricum, Chionanthus Virginica, Chloralum, Chloramphenicolum, Chlorinum, Chloroforum, Chlorpromazinum, Cholera, Cholesterinum, Cholinum, Chromicum Acidum, Chromium Kali Sulphuricum, Chromium Oxydatum, Chromium Sulphuricum, Chrysanthemum Leucanthemum, Chrysarobinum, Cicer Arietinum, Cichorium Intybus, Cichorium Intybus, Flos, Cicuta Maculata, Cicuta Virosa, Cimex Lectularius, Cimicifuga Racemosa, Cina, Cinchona Officinalis, Cinchonium Sulphuricum, Cineraria Maritima, Cineraria Maritima, Succus, Cinnamomum, Cistus Canadensis, Citricum Acidum, Citrus Decumana, Citrus Limonum, Citrus Vulgaris, Clematis Erecta, Clematis Virginiana, Clematis Vitalba, Flos, Clematis Vitalba, Folia, Cobaltum Metallicum, Cobaltum Muriaticum, Cobaltum Nitricum, Coccinella Septempunctata, Cocculus Indicus, Coccus Cacti, Cochlearia Armoracia, Cochlearia Officinalis, Coenzyme A, Coffea Cruda, Coffea Tosta, Colchicinum, Colchicum Autumnale, Colibacillinum, Collinsonia Canadensis, Colocynthinum, Colocynthis, Colostrum, Comocladia Dentata, Conchiolinum, Condurango, Coniinum, Coniinum Bromatum, Conium Maculatum, Convallaria Majalis, Convolvulus Arvensis, Copaiva Officinalis, Corailium Rubrum, Corallorhiza Odontorhiza, Coriaria Ruscifolia, Cornus Alternifolia, Cornus Circinata, Cornus Florida, Cortisone Aceticum, Corydalis Canadensis, Cotyledon Umbilicus, Coumarinum, Crataegus Oxyacantha, Cresolum, Crocus Sativus, Crotalus Cascavella, Crotalus Horridus, Croton Tiglium, Crotonchloralum, Cubeba Officinalis, Cucurbita Citrullus, Cucurbita Pepo. Flos, Cucurbita Pepo, Semen, Culex Musca, Cuphea Petiolata, Cupressus Australis, Cupressus Lawsoniana, Cuprum Aceticum, Cuprum Ammonio-Sulphuricum, Cuprum Arsenicosum, Cuprum Carbonicum, Cuprum Metallicum, Cuprum Muriaticum, Cuprum Nitricum, Cuprum Oxydatum Nigrum, Cuprum Sulphuricum, Curare, Cyclamen Europaeum, Cydonia Vulgaris, Cynara Scolymus, Cynodon Dactylon, Cypripedium Pubescens, Cysteinum, Cytisus Scoparius, Damiana, Daphne Indica, Datura Arborea, Datura Metel, DDT, Delphininum, Derris Pinnata, Dichapetalum, Dictamnus Albus, Digitalinum, Digitalis Purpurea, Digitoxinum, Dioscorea Villosa, Dioscoreinum, Diphtherinum, Diphtherotozinum, Diptherinum, Diptherotoxinum, Dirca Palustris, DNA, Dolichos Pruriens, Doryphora Decemlineata, Draba Verna, Drosera Rotundifolia, DTTAB (Diptheria), Duboisia Myoporoides, Dulcamara, Dulcamara, Flos, Dysentery, E. Coli, Ear, Labyrinth of (inner ear), Ear, Middle, Eberthinum, Echinacea Angustifolia, Echinacea Purpurea, Elaeis Guineensis, Elaps Corallinus, Elaterium, Embryo Suis, Emetinum, Enterotoccinum, Eosinum Natrum, Ephedra Vulgaris, Epigaea Repens, Epilobium Palustre, Epiphegus Virginiana, Equisetum Arvense, Equisetum Hyemale, Eranthis Hyemalis, Erechtites Hieracifolia, Erigeron Canadensis, Eriodictyon Californicum, Erodium, Eryngium Aquaticum, Eryngium Maritimum, Erythraea Centaurium, Eschscholtzia Californica, Eserinum, Etherum, Ethylicum, Ethylum Nitricum, Eucalyptol, Eucalyptus Globulus, Eugenia Caryophyllata, Eugenia Jambosa, Euonymus Atropurpureus, Euonymus Europaeus, Eupatorium Aromaticum, Eupatorium Cannabinum, Eupatorium Perfoliatum, Eupatorium Purpureum, Euphorbia Amygdaloides, Euphorbia Corollata, Euphorbia Cyparissias, Euphorbia Hypericifolia, Euphorbia Lathyris, Euphorbia Pilulifera, Euphorbium Officinarum, Euphrasia Officinalis, Eupion, Eyebright herb, Fagopyrum Esculentum, Fagus Sylvatica, Fagus Sylvatica, Flos, Fel Tauri, Ferrum Aceticum, Ferrum Arsenicicum, Ferrum Bromatum, Ferrum Carbonicum, Ferrum Citricum, Ferrum Cyanatum, Ferrum Iodatum, Ferrum Lacticum, Ferrum Metallicum, Ferrum Muriaticum, Ferrum Pernitricum, Ferrum Phosphoricum, Ferrum Picricum, Ferrum Sulphuricum, Ferrum Tartaricum, Ferula Glauca, Ficus Religiosa, Filix Mas, Foeniculum Vulgare, Folliculinum, Formalinum, Formica Rufa, Formicum Acidum, Fragaria Vesca, Franciscea Uniflora, Fraxinus Americana, Fraxinus Excelsior, Fuchsinum, Fucus Vesiculosus, Fumaria Officinalis, Fumaricum Acidum, Funiculus Umbilicalis Suis, Galanthus Nivalis, Galega Officinalis, Galium Aparine, Gallicum Acidum, Galphimia Glauca, Gambogia, Garlic, Gaultheria Procumbens, Gelsemium Sempervirens, Genista Tinctoria, Gentiana Cruciata, Gentiana Lutea, Gentiana Quinqueflora, Gentianella Amarella, Flos, Geranium Maculatum, Geranium Robertianum, Geum Rivale, Geum Urbanum, Ginkgo Biloba, Glandula Suprarenalis Suis, Glechoma Hederacea, Glonoinum, Glycerinum, Glycogenum, Glycyrrhiza Glabra, Gnaphalium Leontopodium, Gnaphalium Polycephalum, Gnaphalium Uliginosum, Gonotoxinum, Gossypium Herbaceum, Granatum, Graphites, Gratiola Officinalis, Grindelia, Guaco, Guaiacum, Guarea Trichilioides, Guatteria Gaumeri, Gunpowder, Gymnocladus Canadensis, Haematoxylon Campechianum, Haemophilus Infl. B, Hair Bulb, Pilo Sebaceous Zone, HamamelisVirginiana, Haronga Madagas-cariensis, Hedeoma Pulegioides, Hedera Helix, Hekla Lava, Helianthemum Nummularium, Flos, Helianthus Annuus, Heliotropium Peruvianum, Helix Tosta, Helleborus Foetidus, Helleborus Niger, Helleborus Viridis, Heloderma, Helonias Dioica, Hepar Suis, Hepar Sulphuris Calcareum, Hepar Sulphuris Kalinum, Hepatica Triloba, Hepatitis A, Hepatitis B, Hepatitis C, Heracleum Sphondylium, Herpes Zoster, Hippozaeninum, Hippuricum Acidum, Hirudinum, Hi staminum Hydrochloricum, Hoang-Nan, Hoitzia Coccinea, Holarrhena Antidysenterica, Homarus, Hottonia Palustris, Flos, Humulus Lupulus, Hura Brasiliensis, Hura Crepitans, Hydrangea Arborescens, Hydrastininum Muriaticum, Hydrastis Canadensis, Hydrocotyle Asiatica, Hydrocyanicum Acidum, Hydrofluoricum Acidum, Hydrophis Cyanocinctus, Hydrophyllum Virginianum, Hyoscyaminum, Hyoscyaminum, Hydrobromatum, Hyoscyamus Niger, Hypericum Perforatum, Hypothalamus, Iberis Amara, Ichthyolum, Ignatia Amara, Ilex Aquifolium, Ilex Aquifolium, Flos, Ilex Paraguariensis, Illicium Anisatum, Impatiens Glandulifera, Flos, Imperatoria Ostruthium, Indigo, Indium Metallicum, Indolum, Influenzinum, Inula Helenium, lodium, lodoformum, Ipecacuanha, Ipomoea Stans, Iridium Metallicum, Iris Florentina, Iris Foetidissima, Iris Germanica, Iris Tenax, Iris Versicolor, Jacaranda Caroba, Jalapa, Jasminum Officinale, Jasper, Jatropha Curcas, Jatropha Urens, Jequirity, Jonesia Asoca, Juglans Cinerea, Juglans Regia, Juglans Regia, Flos, Juncus Effusus, Juniperus Communis, Juniperus Virginiana, Justicia Adhatoda, Kali Aceticum, Kali Arsenicosum, Kali Bichromicum, Kali Bromatum, Kali Carbonicum, Kali Causticum, Kali Chloricum, Kali Chromicum, Kali Cyanatum, Kali Ferrocyanatum, Kali lodatum, Kali Muriaticum, Kali Nitricum, Kali Oxalicum, Kali Permanganicum, Kali Phosphoricum, Kali Picricum, Kali Silicatum, Kali Sulphuricum, Kali Tartaricum, Kali Telluricum, Kalmia Latifolia, Kamala, Karaka, Karwinskia Humboldtiana, Kino Australiensis, Kousso, Kreosotum, Laburnum Anagyroides, Lac Caninum, Lac Defloratum, Lac Felinum, Lac Matemum, Lac Vaccinum, Lacerta Agilis, Lachesis Mutus, Lachnanthes Tinctoria, Lacticum Acidum, Lactuca Virosa, Lamium Album, Lapis Albus, Lappa Major, Larix Decidua, Flos, Lathyrus Cicera, Lathyrus Sativus, Latrodectus Katipo, Latrodectus Mactans, Laurocerasus, Lecithin granules, Lecithin potenized, Ledum Palustre, Lemna Minor, Leonurus Cardiaca, Lepidium Bonariense, Leptandra Virginica, Lespedeza Capitata, Levico, Levisticum Officinale, Levomepromazinum, Liatris Spicata, Lilium Tigrinum, Limulus, Linaria Vulgaris, Linum Catharticum, Linum Usitatissimum, Lithium Benzoicum, Lithium Bromatum, Lithium Carbonicum, Lithium Muriaticum, Lobelia Cardinalis, Lobelia Erinus, Lobelia Inflata, Lobelia Purpurescens, Lobelia Syphilitica, Lobelinum, Lolium Temulentum, Lonicera Caprifolium, Flos, Lonicera Periclymenum, Lonicera Xylosteum, Lophophytum Leandri, Luesinum, Luffa Operculata, Lupulinum, Lycopersicum Esculentum, Lycopodium Clavatum, Lycopus Virginicus, Lysimachia Nummularia, Lyssin, Lyssinum, Macrotinum, Magnesia Carbonica, Magnesia Muriatica, Magnesia Oxydata, Magnesia Phosphorica, Magnesia Sulphurica, Magnesium Metallicum, Magnolia Glauca, Magnolia Grandiflora, Malaria Off, Malus Pumila, Flos, Mancinella, Mandragora Officinarum, Manganum Aceticum, Manganum Carbonicum, Manganum Metallicum, Manganum Muriaticum, Manganum Oxydatum Nativum, Manganum Oxydatum Nigrum, Manganum Phosphoricum, Manganum Sulphuricum, Mangifera Indica, Marrubium Vulgare, Matico, Matthiola Graeca, Medorrhinum (Gonorrheal virus), Medulla Ossis Suis, Medusa, Melastoma Ackermani, Melilotus Alba, Melilotus Officinalis, Melissa Officinalis, Menispermum Canadense, Mentha Piperita, Mentha Pulegium, Mentha Viridis, Mentholum, Menyanthes Trifoliata, Mephitis Mephitica, Mercurialis Perennis, Mercurius Aceticus, Mercurius Auratus, Mercurius Bromatus, Mercurius Corrosivus, Mercurius Cum Kali Iodatus, Mercurius Cyanatus, Mercurius Dulcis, Mercurius Iodatus Flavus, Mercurius Iodatus Ruber, Mercurius Methylenus, Mercurius Nitricus, Mercurius Praecipitatus Albus, Mercurius Praecipitatus Ruber, Mercurius Solubilis, Mercurius Sulphocyanatus, Mercurius Sulphuratus Ruber, Mercurius Sulphuricus, Mercurius Vivus, Methylene Blue, Mezereum, Millefolium, Mimosa Pudica, Mimulus Guttatus, Flos, Mitchella Repens, Momordica Balsamina, Mononucleosis, Monotropa Uniflora, Morbillinum (Measles), Moschus, Mucosa Nasalis Suis, Mullein Essence, Murex Purpurea, Muriaticum Acidum, Musa Sapientum, Mygale, Myosotis Arvensis, Myrica Cerifera, Myristica Sebifera, Myrrha, Myrtus Communis, Nabalus Serpentarius, Nadidum, Naja Tripudians, Naphthalinum, Narceinum, Narcissus, Pseudo-, Narcissus, Narcotinum, Nasturtium Aquaticum, Natrum Arsenicicum, Natrum Bicarbonicum, Natrum Bromatum, Natrum Carbonicum, Natrum Fluoratum, Natrum Hypochlorosum, Natrum Lacticum, Natrum Muriaticum, Natrum Nitricum, Natrum Nitrosum, Natrum Oxalaceticum, Natrum Phosphoricum, Natrum Pyruvicum, Natrum Salicylicum, Natrum Silicofluoricum, Natrum Sulphuratum, Natrum Sulphuricum, Natrum Sulphurosum, Negundo, Nepenthes, Nepeta Cataria, Niccolum Carbonicum, Niccolum Metallicum, Niccolum Sulphuricum, Nicotinamidum, Nicotinum, Nitri Spiritus Dulcis, Nitricum Acidum, Nitrogenum Oxygenatum, Nitromuriaticum Acidum, Nosode Kit, Nosode-Select your own, Nuclear Radiation, Nuphar Luteum, Nux Moschata, Nux Vomica, Nymphaea Odorata, Ocimum Basilicum, Ocimum Canum, Ocimum Sanctum, Oenanthe Crocata, Oenothera Biennis, Olea Europaea, Flos, Oleander, Oleum Animale, Oleum Carvi, Oleum Morrhuae, Oleum Ricini, Oleum Santali, Olibanum, Oniscus, Ononis Spinosa, Onopordum, Onosmodium Virginianum, Oophorinum, Opuntia Vulgaris, Orchitinum, Oreodaphne Californica, Origanum Majorana, Ornithogalum Umbellatum, Ornithogalum Umbellatum, Flos, Oroticum Acidum, Oscillococcinum, Osmium Metallicum, Ostrya, Ova Tosta, Ovi Gallinae Pellicula, Oxalicum Acidum, Oxalis Acetosella, Oxydendrum Arboreum, Oxytropis Lambertii, Paeonia Officinalis, Palladium Metallicum, Paloondo, Pancreas Suis, Pancreatinum, Paraffinum, Parathormonum, Parathyroid, Paratyphoidinum B, Pareira Brava, Parietaria Officinalis, Paris Quadrifolia, Paronichia Illecebrum, Parotidinum (Mumps), Parthenium, Passiflora Incarnata, Pastinaca Sativa, Paullinia Pinnata, Paullinia Sorbilis, Pecten, Pediculus Capitis, Penicillinum, Penthorum Sedoides, Pepsinum, Perhexilinum, Persea Americana, Pertussinum (Whooping Cough), Petiveria Tetrandra, Petroleum, Petroselinum Sativum, Phallus Impudicus, Phaseolus, Phellandrium Aquaticum, Phenacetinum, Phenobarbitalum, Phloridzinum, Phosphoricum Acidum, Phosphorus, Physalis Alkekenge, Physotigma Venenosum, Phytolacca Decandra, Pichi, Picricum Acidum, Picrotoxinum, Pilocarpinum, Pilocarpinum Muriaticum, Pilocarpinum Nitricum, Pilocarpus, Pimenta Officinalis, Pimpinella Saxifraga, Pinus Lambertiana, Pinus Sylvestris, Pinus Sylvestris, Flos, Piper Methysticum, Piper Nigrum, Piperazinum, Piscidia Erythrina, Pituitarum Posterium, Pix Liquida, Placenta Totalis Suis, Plague, Plantago Major, Platanus, Platinum Metallicum, Platinum Muriaticum, Plectranthus Fruticosus, Plumbago Littoralis, Plumbum Aceticum, Plumbum Carbonicum, Plumbum Chromicum, Plumbum Iodatum, Plumbum Metallicum, Pneumococcinum, Podophyllinum, Podophyllum Peltatum, Polio, Polygonum Punctatum, Polygonum Sagittatum, Polyporus Officinalis, Polyporus Pinicola, Populus Candicans, Populus Tremula, Flos, Populus Tremuloides, Potentilla Anserina, Pothos Foetidus, Primula Obconica, Primula Veris, Primula Vulgaris, Proteus Bulgaris, Proteus Vulgaris, Prunus Cerasifera, Flos, Prunus Padus, Prunus Spinosa, Prunus Virginiana, Psorinum, Ptelea Trifoliata, Pulex Irritans, Pulsatilla Niger, Pulsatilla Nuttalliana, Pyrethrum Parthenium, Pyridoxinum Hydrochloricum, Pyrogenium-sepsis, Pyrus Americana, Quassia Amara, Quebracho, Quercus Glandium Spiritus, Quercus Robur, Quercus Robur, Flos, Quillaja Saponaria, Radium Bromatum, Ranunculus Acris, Ranunculus Bulbosus, Ranunculus Ficaria, Ranunculus Glacialis, Ranunculus Repens, Ranunculus Sceleratus, Raphanus Sativus, Ratanhia, Rauwolfia Serpentina, Reserpinum, Resina Laricis, Resorcinum, Rhamnus Californica, Rhamnus Cathartica, Rhamnus Frangula, Rhamnus Purshiana, Rheum Officinale, Rhodium Metallicum, Rhododendron Chrysanthum, Rhus Aromatica, Rhus Diversiloba, Rhus Glabra, Rhus Toxicodendron, Rhus Venenata, Riboflavinum, Ricinus Communis, RNA, Robinia Pseudoacacia, Rock Water, Rosa Canina, Rosa Canina, Flos, Rosa Damascena, Rosmarinus Officinalis, Rubella (German Measles), Rubeola (Measles), Rubia Tinctorum, Rumex Acetosa, Rumex Crispus, Rumex Obtusifolius, Russula Foetens, Ruta Graveolens, Sabidilla, Sabal Serrulata, Sabina, Saccharinum, Saccharum Lactis, Saccharum Officinale, Salicinum, Salicylicum Acidum, Salix Alba, Salix Nigra, Salix Purpurea, Salix Vitellina, Flos, Salmonella, Salol, Salvia Officinalis, Samarskite, Sambucus Canadensis, Sambucus Nigra, Sanguinaria Canadensis, Sanguinarinum Nitricum, Sanicula, Santoninum, Saponaria Officinalis, Saponinum, Sarcode-Select your own organ remedy, Sarcolacticum Acidum, Sarracenia Purpurea, Sarsaparilla, Sassafras Officinale, Scammonium, Scarlatinum, Secale-Ergot Schinus Molle, Scilla Maritima, Scleranthus Annuus, Flos, Scolopendra, Scolopendrium Vulgare, Scopolaminum Hydrobromidum, Scrophularia Nodosa, Scutellaria Lateriflora, Secale Cornutum, Secale-Ergot, Sedum Acre, Selenium Metallicum, Sempervivum Tectorum, Senecio Aureus, Senecio Jacobaea, Senega Officinalis, Senna, Sepia, Serum Anguillae, Serum Anticolibacillaire, Serum De Yersin, Serum Equi, Shigella, Silica Marina, Silicea, Silphium Laciniatum, Sinapis Alba, Sinapis Arvensis, Flos, Sinapis Nigra, Sinusitisinum, Sium Latifolium, Skatolum, Skookum Chuck, Slag, Solaninum, Solanum Arrebenta, Solanum Carolinense, Solanum Mammosum, Solanum Nigrum, Solanum Oleraceum, Solanum Tuberosum, Solidago Virgaurea, Sparteinum Sulphuricum, Spigelia Anthelmia, Spigelia Marilandica, Spilanthes Oleracea, Spinacia, Spiraea Ulmaria, Spiranthes Autumnalis, Spongia Encephalitis, Spongia Tosta, Stachys Betonica, Stannum Iodatum, Stannum Metallicum, Staphyloccoccus Aureus, Staphylococcinum, Staphylotoxinum, Staphysagria, Stellaria Media, Sterculia Acuminata, Stibium Metallicum, Sticta Pulmonaria, Stigmata Maidis, Stillingia Sylvatica, Stramonium, Streptococcinum, Strontium Bromatum, Strontium Carbonicum, Strontium Nitricum, Strophanthus Hispidus, Strophanthus Sarmentosus, Strychninum, Strychinum Arsenicicum, Strychinum Nitricum, Strychninum Phosphoricum, Strychninum Sulphuricum, Succinicum Acidum, Succinum, Sulphanilamidum, Sulphonalum, Sulphur, Sulphur Hydrogenisatum, Sulphur Iodatum, Sulphuricum Acidum, Sulphurosum Acidum, Sumbul, Symphoricarpus Racemosus, Symphytum Officinale, Syphilinum (Luesinum), Syzygium Jambolanum, Tabacum, Tamus Communis, Tanacetum Vulgare, Tanghinia Venenifera, Tannicum Acidum, Taraxacum Officinale, Taraxacum Officinale, Radix, Tarentula Cubensis, Tarentula Hispana, Tartaricum Acidum, Taxus Baccata, Tellurium Metallicum, Teplitz, Terebinthina, Tetanotoxinum, Tetradymite, Teucrium Marum, Teucrium Scorodonia, Thallium Metallicum, Thaspium Aureum, Thea Sinensis, Theobrominum, Theridion, Thiaminum Hydrochloricum, Thioproperazinum, Thiosinaminum, Thlaspi Bursa-Pastoris, Thuja Lobbi, Thuja Occidentalis, Thymolum, Thymus Serpyllum, Thyroidinum, Tilia Europaea, Titanium Metallicum, Tongo, Tormentilla, Torula Cerevisiae, Toxicophis Pugnax, Tradescantia Diuretica, Tribulus Terrestris, Trifolium Pratense, Trifolium Repens, Trillium Pendulum, Trimethylaminum, Triosteum Perfoliatum, Triticum Repens, Tropaeolum Majus, Tuberculinum, Tuberculinum Residuum, Tussilago Farfara, Tussilago Fragrans, Tussilago Petasites, Typhoidinum, Ulex Europaeus, Flos, Ulmus Fulva, Ulmus Procera, Flos, Upas Tieute, Uranium Nitricum, Urea, Uricum Acidum, Urtica Crenulata, Urtica Dioica, Urtica Urens, Usnea Barbata, Ustilago Maidis, Uva-Ursi herb, Uva-Ursi, V.A.B.-BCG, Vaccinium Myrtillus, Vaccinotoxinum, Valeriana Officinalis, Vanadium Metallicum, Varicella enus Mercenaria (Chicken Pox), Variolinum (Smallpox), Veratrinum, Veratrum Album, Veratrum Nigrum, Veratrum Viride, Verbascum Thapsus, Verbena Hastata, Verbena Officinalis, Verbena Officinalis, Flos, Veronica Beccabunga, Veronica Officinalis, Vesicaria, Vespa Crabro, Viburnum Opulus, Viburnum Prunifolium, Vinca Minor, Viloa Odorata, Viola Tricolor, Vipera Berus, Viscum Album, Vitamin B12, Vitamin K, Vitis Vinifera, Flos, Wiesbaden, Wyethia Helenioides, X-Ray, Xanthoxylum Fraxineum, Xerophyllum Asphodeloides, Yohimbinum, Yucca Filamentosa, Zincum Aceticum, Zincum Bromatum, Zincum Carbonicum, Zincum Cyanatum, Zincum Gluconicum, Zincum Iodatum, Zincum Metallicum, Zincum Muriaticum, Zincum Oxydatum, Zincum Phosphoratum, Zincum Picricum, Zincum Sulphuricum, Zincum Valerianicum, Zingiber Officinale, and combinations thereof.

Flower essences can also be included in cannabinoid compositions prepared according to the invention. Examples of suitable flower essences include, but are not limited to, Acacia, Actaea, Agrimony, Alpine Lily, Angel's Trumpet, AloeVera, Angelica, Basil, Apricot, Arnica Beech, Aspen, Avocado, Beech, Bee Balm, Black Cohosh, Baby Blue Eyes, Black-Eyed Susan, Blackberry, Bloodroot, Calendula, Bleeding Heart, California Fuchsia, California Pitcher Plant, Borage, Buttercup, California Wild Rose, California Poppy, CallaLily, Cerato, Canyon Dudleya, Chamomile, Cayenne, Cedar, Chaparral, Centaury, Centaurium erythraea or Centaurium umbellatum, Cerato, Cherry Plum, Chestnut Bud, Corn, Dandelion, Chicory, Cinquefoil, Coffee, Coreopsis, Crab Apple, Chrysanthemum, Clematis, Desert Dandelion, Deerbrush, Cosmos, Dill, Elm, Evening Primrose, Dogwood, Easter Lily, Eucalyptus, Fairy Lantern, Echinacea, Fawn Lily, Fig, Filaree, Gentian, Goldenrod, Forget-Me-Not, Golden Ear Drops, Golden Yarrow, Fuchsia, Garlic, Gorse, Honeysuckle, Heather, Hornbeam, Hibiscus, Hound's Tongue, Holly, Impatiens, Indian Paintbrush, Larch, Lily, Indian Pink, Larkspur, Iris, Iris douglasiana/Iris versicolor, Lady's Slipper, Cypripedium parviflorum/Cypripedium reginae, Lotus, Lavender, Love-Lies-Bleeding, Mariposa Lily, Madia, Magnolia, Milkweed, Mallow, Mimulus, Manzanita, Morning Glory, Motherwort, Mountain Pennyroyal, Mustard, Mountain Pride, Nasturtium, Mugwort, Nicotiana, Noni, Oak, Olive, Pine, Orange, Oregon Grape, Pansy, Passion Flower, Pear, Petunia, Pink Angel's Trumpet, Pink Monkeyflower, Penstemon, Pink Yarrow Achillea millefolium var. rubra, Peppermint, Poison Oak, Pomegranate, Queen Anne's Lace, Pretty Face, Quince, Purple Monkeyflower, Rabbitbrush, Quaking Grass, Red Chestnut, Red clover, Rescue Remedy, Rock Rose, Sacred Datura, Sagebrush, Scarlet Pimpernel, Rock Water Solarized spring water, Saguaro, Rosemary, Rose, Saint John's Wort, Scarlet Monkeyflower, Shasta Daisy, Scleranthus, Shooting Star, Scotch Broom, Snapdragon, Squash, Self-Heal, Star of Bethlehem, Star Thistle, Sweet Chestnut, Star Tulip, Strawberry, Sun Cup, Sweet Pea, Sticky Monkeyflower, Tansy, Sunflower, Thyme, Tiger Lily, Trillium, Violet, Walnut, Trumpet Vine, Vervain, Water Lily, Water Violet, Vine, White Chestnut, Wild Oat, Wild Rose, Yellow Star Tulip, Willow, Yerba Santa, Yarrow, Yucca, Zinnia, and combinations thereof.

### D. Oral Formulations

The formulations of the present disclosure include compositions comprising an intoxication-mediating active agent (e.g., CBD and/or black pepper) and at least one excipient suitable for use in oral dosage forms. The formulations can be provided in any suitable form, including but not limited to a liquid form, a gel form, a semi-liquid (e.g., a liquid, such as a viscous liquid, containing some solid) form, a semi-solid (a solid containing some liquid) form, or a solid form. The oral formulation can be adapted for transmucosal delivery via the buccal route, labial route, gingival route or sublingual route or to be swallowed. Exemplary oral formulations include buccal gel, film, gum, chewable tablet or lozenge, orally disintegrating tablet, hard lozenge or lollipop, and liquids (e.g., solution or suspension). The oral formulations are prepared by mixing the active ingredients with one or more inactive ingredients, such as pharmaceutically acceptable excipients. In some cases, the one or more pharmaceutically acceptable excipients can be selected from sugar alcohols such as sorbitol, xylitol, maltitol, mannitol, isomalt, erythritol and combinations thereof, hydrocolloids, such as gelatin, gum acacia, carob gum, carrageenan, ghatti gum, guar gum, karaya gum, pectin, tragacanth gum, locust bean gum, xanthan gum and combinations thereof, and emulsifiers, surfactants, colorants, flavorants, taste modifiers, buffering agents, pH adjusting agents, stabilizers, and sweeteners. Formulations can be provided in, for example, a slow-release form, a non-slow-release form, a sustained release form, or a non-sustained-release form.

In some cases, the formulation is a tablet, caplet, capsule, including soft gelatin capsules, microcapsule, and lozenge. Tablets can further comprise suitable binders, lubricants, diluents, disintegrating agents, colorants, flavoring agents, flow-inducing agents, and melting agents. In some embodiments the oral formulation is a compressed tablet comprising ground peppercorns or a suitable extract therefrom along with cannabidiol with a quantity of about 0.01 mg to 500 mg, along with an appropriate excipient(s) to facilitate tablet compression. In some embodiments the oral formulation is a candy-style lozenge comprising ground peppercorn or suitable extract therefrom, along with cannabidiol at a quantity of 0.01 to 500 mg along with appropriate lozenge ingredients. The term “candy-style lozenge” as used herein refers to a solid preparation that is intended to dissolve or disintegrate slowly in the mouth, keeping the active ingredients in contact with the oral cavity for an extended period of time. A candy-style lozenge can be made by compression or molding, include one or more of flavorings, colorings, and sweeteners. A candy-style lozenge can be hard, soft or chewable, and include forms such as lozenges include lollipops and gummies.

In some embodiments the oral formulation is an oral solution. In some embodiments the oral solution is packaged in order to facilitate the delivery of single doses, such as, for example, with a medicine dropper or in a spray/spritz-style package.

The content of peppercorn, peppercorn substitute, or extract thereof can be varied based on the terpenoid profile of the plant tissue or extract. In some cases, the oral formulation includes about 100 mg black pepper extract or about 1 gm of ground black pepper drupe. In some embodiments the oral formulation includes coated whole peppercorns. The coating can include 0.01 mg to 500 mg CBD.

In some embodiments, the formulations of the present invention are made by combining a CBD-sugar alcohol (or sugar) intermediate with the one more excipients, and optionally black pepper or an extract thereof. The sugar alcohol formulated intermediate may be prepared by the procedure described in the Examples

Suitable dosages of the active agent for use in the methods of the present disclosure will depend upon a number of factors including, for example, age and weight of an subject being treated, frequency of cannabis use, severity of the unpleasant effect/condition to be ameliorated, specific compounds included in the composition to be used, nature of a composition, route of administration and combinations thereof. Ultimately, a suitable dosage can be readily determined by a skilled artisan (e.g., physician or pharmacist). The subject can be administered a low dosage initially. The dose can be increased until sufficient to prevent or ameliorate unpleasant effects experienced by a user of cannabis (e.g., anxiety). Alternatively, one skilled in the art can begin with a high dosage that can be decreased until reaching a minimum dosage needed to prevent or ameliorate the unpleasant effects experienced by a user of cannabis. In some cases, the unpleasant effect to be ameliorated is psychotic-like, depressive and anxiety symptoms.

The following Examples, which illustrate an embodiment of the present disclosure, should not be construed as to narrow its scope.

### EXAMPLES

### CBD-Sugar or Sugar Alcohol Intermediate

i. Prepare a Concentrated Oil (CO) Formulate:

Calculate quantities of the CBD containing oil to add in order to achieve a desired potency/concentration. Heat oil to workable fluidity (e.g., 50-60° C.). The CO formulation can include one or more other non-intoxicating cannabinoids (e.g., CBN and/or CBG).

ii. Prepare a Solvent Formulate:

Calculate quantities of CO Formulate and solvent (ethanol) needed. Heat CO Formulate to workable fluidity (50-60° C.). Slowly adding each component in an appropriately sized vessel until reaching setpoints determined by formulation calculations (±1 g desired mass). Typical ratio is 1:1, i.e., 1 part CO Formulate to 1 part 200 proof ethanol, or similar solvent or mixture thereof. The Ethanol Formulated is sonicated in a bath sonicator.

In some cases, the Ethanol Formulate is homogenized with a probe sonicator. A taller, thinner vessel is preferred over a shorter, wider one for sonication. Sanitize probe by wiping with 200 proof ethanol before sonicating. Run sonicator per manufacturer's recommendations according to substrate volume, viscosity, composition, etc. Place vessel containing Ethanol Formulate in ice bath to keep solution cool during sonication. (e.g., dry ice and ethanol or water when available, or mixture of ice, water, and rock salt). Set temperature probe and sonicator probe in vessel. The tip of the sonicator probe should be about ⅓ from top of Ethanol Formulate. Ensure the sonicator probe never touches any glass or metal.

During the sonication process, the vessel is watched to verify it does not move or touch sonication probe. If temperature exceeds setpoint, the program should automatically pause. When this happens, wait for solution to reach below 30° C. and restart program. When finished, remove vessel from sonicator. Clean sonicator and temperature probes. Dispose of ice bath. The homogenized Ethanol Formulate is filtered (e.g., a 200 nm filter).

iii. Prepare a Sugar Alcohol (or Sugar) Formulate:

Melt sugar or sugar alcohol. It takes about 55 minutes to melt 800 g isomalt in one 9″×13″ Pyrex baking dish. Larger quantities of isomalt can take several hours to melt completely. Using figures from Formulation calculations to provide a specific dose of CBD (e.g., 200-400 mg/per dosage unit) measure isomalt into each Pyrex baking dish until total amount is achieved. Heat isomalt in oven until it becomes a uniform, clear liquid. Remove from oven immediately to avoid discoloration and off flavors (a burnt flavor).

While isomalt is heating, prepare mixing equipment and additional components (Quillaja extract (emulsifier) and gum Arabic (hydrocolloid)). A mixing container (such as a silicone pot or stainless pot) is cleaned and sanitized. When added to the isomalt, the quillaja extract will cause foaming. A suitably sized container will be at least 3 or 4 times the volume of the isomalt. Pre-measure quillaja extract per Formulation calculations in small beaker. Pre-measure gum Arabic in small beaker. When the isomalt is a clear, hot liquid, remove from oven and empty into silicone pot. While stirring, pour quillaja extract and half of the gum Arabic into isomalt. Keep stirring until foaming subsides and mixture is uniform in color.

Per Formulation calculations, add pre-determined amount of Ethanol Formulate. One method is to tare scale with vessel containing Ethanol Formulate. Pour Ethanol Formulate until scale shows the desired quantity has been removed. It is better to over-pour than to under-pour as the concentration can be reduced in the Final Formulation as needed. Ethanol will start evaporating once it hits hot isomalt, thereby removing the ethanol. This step should be done in a fume hood whenever possible. Keep stirring until color is uniform.

While hot, pour the molten mixture of isomalt, quillaja extract, gum Arabic, and cannabis oil mixture out onto suitable surface and spread, while hot, as thin of a layer as possible. Allow to cool to room temperature to solidify. The cooling process can be accelerated by cooling on and/or under dry ice.

iv. Prepare the Sugar Alcohol (or Sugar) Intermediate

Once the isomalt formulate is completely cooled (no longer tacky and at room temperature) it can be broken into smaller pieces, aka “shards”. The hardness can be tested with a clean utensil—if the isomalt formulate does not allow deformation with a pointed utensil, it is ready to be broken. Cover the isomalt formulate to prevent isomalt shards from flying during the breaking process. Use hammer or hands or other suitable tools to break the solid sheet of isomalt formulate into small pieces.

Add the isomalt formulate shards to a collection vessel. Get a final weight of isomalt formulate added. Add remaining gum Arabic as a lubricant for size reduction. Total gum Arabic content is 1-2% of the mass of the isomalt formulate. Mix as well as possible to ensure the maximum amount of isomalt formulate is coated with gum Arabic powder. Record the amount of gum Arabic added.

Mill isomalt formulate shards into a powder of desired particle size using a mill or other appropriate equipment. The bulk powder was subjected to mechanical separation and filtering to provide a powder having a particle size of no greater than 600 microns.

### Formulations Using the CBD-Sugar or Sugar Alcohol Intermediate

The resulting intermediate is a water-soluble CBD powder that can be combined with ground or whole peppercorns, or an extract thereof, and suitable tabletting excipients using conventional amounts, equipment, and methods for direct compression of a specific dose of CBD. Alternatively, the soluble CBD powder can be combined with at least one extract or powder from peppercorn as active ingredients of a solution or suspension (e.g., an oral spray) with one or more inert ingredients, selected from the group consisting of organic solvents, emulsifiers, flavor modifiers, and antifoam agents, in oil or water. The solution or suspension can be dispensed from an atomizer or nebulizer, or other means for producing droplets of liquid.

In some embodiments the oral formulations are fortified with specific terpenes known to decrease the sensation or the negative experience associated with acute cannabis intoxication.

In some embodiments the oral formulation is chewable. In some embodiments the oral formulation is a time released formulation. In some embodiments the cannabidiol has been further formulated to facilitate buccal delivery.

All formulations described are to be made in accordance with methods known to those having skill in the art and utilizing standard excipients, diluents, and processes.

While the present disclosure includes many embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

With respect to the above, it is to be understood that the invention is not limited in its application to the details of construction and to the arrangement of the components listed or the steps set forth in the description. The various apparatus and methods of the disclosed invention are capable of other embodiments, and of being practiced and carried out in various ways that would be readily known to those skilled in the art, given the present disclosure. Further, the terms and phrases used herein are for descriptive purposes and should not be construed as in any way limiting.

As such, those skilled in the art will appreciate that the conception upon which this disclosure is based may be utilized as a basis for designing other inventions with similar properties. It is important therefore that the embodiments, objects, and claims herein, be regarded as including such equivalent construction and methodology insofar as they do not depart from the spirit and scope of the present invention.

What is claimed is:

1. An oral formulation for mitigating an effect of acute cannabis intoxication comprising:
a peppercorn, a peppercorn substitute, or an extract thereof;
a pharmaceutically acceptable excipient; and
optionally CBD.

2. The oral formulation of claim 1, wherein the peppercorn is a black peppercorn.

3. The oral formulation of claim 1, comprising about 0.01 mg to 500 mg CBD.

4. The oral formulation of claim 1, further comprising a non-intoxicating cannabinoid.

5. The oral formulation of claim 1, comprising a quantity of peppercorn, peppercorn substitute or extract thereof to achieve a predetermined amount of terpenoid, optionally a predetermined amount of β-caryophyllene.

6. The oral formulation of claim 1, wherein the formulation is an oral spray.

7. The oral formulation of claim 1, wherein the formulation is a compressed tablet.

8. The oral formulation of claim 1, wherein the formulation is a candy-style lozenge.

9. The oral formulation of claim 1, further containing at least one sugar alcohol.

10. The oral formulation of claim 1, further comprising a hydrocolloid selected from the group consisting of gelatin, gum acacia, carob gum, carrageenan, ghatti gum, guar gum, karaya gum, pectin, tragacanth gum, locust bean gum, xanthan gum and combinations thereof.

11. The oral formulation of claim 1, wherein the pharmaceutically acceptable excipient is selected from the group consisting of emulsifiers, surfactants, colorants, flavorants, taste modifiers, buffering agents, pH adjusting agents, stabilizers, sweeteners and combinations thereof.

12. A method of mitigating an effect of acute cannabis intoxication in a subject in need thereof, the method comprising administering to the subject an oral formulation comprising:
a peppercorn, a peppercorn substitute, or an extract thereof;
a pharmaceutically acceptable excipient; and
optionally CBD.

13. The method of claim 12, wherein the peppercorn is a black peppercorn.

14. The method of claim 12, wherein the oral formulation comprises about 0.01 mg to 500 mg CBD.

15. The method of claim 12, wherein the oral formulation comprises a quantity of peppercorn, peppercorn substitute or extract thereof to achieve a predetermined amount of terpenoid, optionally a predetermined amount of pinene, piperine, or β-caryophyllene.

16. The method of claim 12, wherein the oral formulation is administered as an oral spray, a compressed tablet, or a candy-style lozenge.

17. The method of claim 12, wherein the oral formulation further comprises at least one sugar alcohol.

18. The method of claim 12, wherein the oral formulation further comprises a hydrocolloid selected from the group consisting of gelatin, gum acacia, carob gum, carrageenan, ghatti gum, guar gum, karaya gum, pectin, tragacanth gum, locust bean gum, xanthan gum and combinations thereof.

19. The method of claim 12, wherein the pharmaceutically acceptable excipient is selected from the group consisting of emulsifiers, surfactants, colorants, flavorants, taste modifiers, buffering agents, pH adjusting agents, stabilizers, sweeteners and combinations thereof.

20. The method of claim 12, wherein the oral formulation is administered after the subject has used cannabis or administered concurrently with cannabis use.
